SEQUENCE LISTING (2 sequences)

SEQ ID NO: 1 - DNA, 24 bp, Artificial Sequence
  Synthetic Sequence
cgggtgggagcagatcttattgag

SEQ ID NO: 2 - DNA, 24 bp, Artificial Sequence
  Synthetic Sequence
cgggtgggagcagatcttattgag

(19) United States
(12) Patent Application Publication
(10) Pub. No.: US 2023/0338288 A1
(43) Pub. Date: Oct. 26, 2023

(54) ASYMMETRIC CHARGED VESICLES AND METHODS OF PREPARING AND USE THEREOF

(71) Applicant: The Research Foundation for the State University of New York, Albany, NY (US)

(72) Inventors: Bingchen Li, Stony Brook, NY (US); Erwin London, Stony Brook, NY (US)

(21) Appl. No.: 18/015,807

(22) PCT Filed: Jul. 14, 2021
(86) PCT No.: PCT/US2021/041563

§ 371 Date: Jan. 12, 2023

Related U.S. Application Data
(60) Provisional application No. 63/068,807, filed on Aug. 21, 2020.
(60) Provisional application No. 63/051,635, filed on Jul. 14, 2020.

(51) Int. Cl. A61K 9/127; C12N 15/88; A61K 31/704; A61K 47/28; A61K 47/24; A61K 51/12; A61K 49/00; A61K 9/107; A61K 47/40

(52) U.S. Cl. CPC A61K 9/127; C12N 15/88; A61K 31/704; A61K 47/28; A61K 47/24; A61K 9/1277; A61K 51/1234; A61K 49/0084; A61K 9/1075; A61K 47/40

(57) ABSTRACT

The present disclosure relates to one or more charged vesicles, each including: a bilayer of lipids forming a shell, wherein the bilayer of lipids includes an inner layer of lipids and an outer layer of lipids, wherein the inner layer of lipids and the outer layer of lipids are different, and wherein the bilayer is characterized by having an asymmetric charge distribution; and an interior portion of the shell configured to entrap a drug. The present disclosure further relates to methods of using and making an asymmetrical vesicle as well as kits related thereto.

### CROSS-REFERENCE TO RELATED APPLICATIONS

The present disclosure claims priority or the benefit under 35 U.S.C. § 119 to U.S. Provisional Application Nos. 63/051,635 filed Jul. 14, 2020, and 63/068,807 filed Aug. 21, 2020, both of which are fully incorporated herein by reference.

### GOVERNMENT SUPPORT

This invention was made with government support under DMR 1709035 awarded by the National Science Foundation. The U.S. Government has certain rights in the invention.

### REFERENCE TO A SEQUENCE LISTING

This application contains a Sequence Listing in computer readable form, which is incorporated herein by reference.

### FIELD OF THE INVENTION

This disclosure is in the field of chemical engineering and relates to the formation of vesicles including lipid bilayers. More specifically, this invention relates to the formation of asymmetric charged vesicles such as by methods for the substitution and exchange of membrane lipids by way of cyclodextrin-lipid complexes. The asymmetric charged vesicles and methods of the present disclosure can be used, for example, in the efficient substitution of one or more outer-leaflet lipids with one or more lipids bound to cyclodextrin-lipid complexes, and/or the use of asymmetric charged vesicles for drug or biomolecule delivery.

### BACKGROUND

Chemotherapeutic drugs and biomedicines should be efficiently delivered to their target. This may be an especially important issue for charged drugs such as doxorubicin, used for cancer treatment (See e.g., Dahm, R. Friedrich Miescher and the discovery of DNA. Developmental Biology 278, 274-288 (2005)), and biomolecules such as RNAs that can be used therapeutically to interfere with gene expression, an approach useful for otherwise undruggable targets (See e.g., Elliott, D. & Ladomery, M. Molecular Biology of RNA. (Oxford University Press, 2017) and Cech, T. R. & Steitz, J. A. The Noncoding RNA Revolution—Trashing Old Rules to Forge New Ones. Cell 157, 77-94 (2014)). As with many drugs, these drug molecules should be presented at relatively high concentrations at specific targets (See e.g., Boccaletto, P. et al. MODOMICS: a database of RNA modification pathways. 2017 update. Nucleic Acids Res 46, D303—D307 (2018) and Albretsen, C., Kalland, K.-H., Haukanes, B.-I., Havarstein, L.-S. & Kleppe, K. Applications of magnetic beads with covalently attached oligonucleotides in hybridization: Isolation and detection of specific measles virus mRNA from a crude cell lysate. Analytical Biochemistry 189, 40-50 (1990)). Direct delivery of a drug can result in poor biodistribution and pharmacokinetics, and so problematically result in unacceptable off-target side effects, short circulation times, drug breakdown and clearance (See e.g., Biscontin, A. et al. New miRNA labeling method for bead-based quantification. BMC Mol Biol 11, 44 (2010)).

To avoid such problems, trapping drugs within liposomes has demonstrated many advantages. Liposomes (lipid vesicles) are dispersions of membrane lipids in which a lipid bilayer surrounds an aqueous lumen. The advantages of using liposomes for drug delivery include the ability to trap many types of drugs within their lipid bilayer or aqueous lumen, easy manufacturing procedures to maintain drug bioactivity, high loading of drug to minimize the dosage needed, the ability to use multi-dosing to maintain an effective drug concentration, the ability to target specific cells, and biocompatibility with long circulation times (See e.g., Adams, N. M. et al. Comparison of Three Magnetic Bead Surface Functionalities for RNA Extraction and Detection. ACS Appl. Mater. Interfaces 7, 6062-6069 (2015); Chandrasekaran, A. R. et al. Cellular microRNA detection with miRacles: microRNA-activated conditional looping of engineered switches. Science Advances 5, eaau9443 (2019); Flora, P. et al. Sequential Regulation of Maternal mRNAs through a Conserved cis-Acting Element in Their 3′ UTRs. Cell Reports 25, 3828-3843.e9 (2018); and Chandrasekaran, A. R., Levchenko, O., Patel, D. S., Maclsaac, M. & Halvorsen, K. Addressable configurations of DNA nanostructures for rewritable memory. Nucleic Acids Res 45, 11459-11465 (2017)).

Work has been done to study the relationship between liposomal lipid properties and the efficacy of drug delivery in different types of cells (See e.g., Chandrasekaran, A. R. et al. Cellular microRNA detection with miRacles: microRNA-activated conditional looping of engineered switches. Science Advances 5, eaau9443 (2019) and Gillespie, J. J., Johnston, J. S., Cannone, J. J. & Gutell, R. R. Characteristics of the nuclear (18S, 5.8S, 28S and 5S) and mitochondrial (12S and 16S) rRNA genes of Apis mellifera (Insecta: Hymenoptera): structure, organization, and retrotransposable elements. Insect Mol Biol 15, 657-686 (2006)). However, the relationship between the lipid properties such as charge, and the drug-loading ability of vesicles has not been fully studied. Charge is an important parameter influencing molecular delivery to cells (See e.g., Decatur, W. A. & Schnare, M. N. Different Mechanisms for Pseudouridine Formation in Yeast 5S and 5.8S rRNAs. Mol Cell Biol 28, 3089-3100 (2008)). Cationic lipid vesicles can be used for delivery of molecules to cultured cells due to their ability to bind to the cell membrane, which facilitates endocytosis, membrane fusion, and endosomal escape (See e.g., Taoka, M. et al. Landscape of the complete RNA chemical modifications in the human 80S ribosome. Nucleic Acids Res 46, 9289-9298 (2018); Wang, X. et al. m6A-dependent regulation of messenger RNA stability. Nature 505, 117-120 (2014); and Sloan, K. E. et al. Tuning the ribosome: The influence of rRNA modification on eukaryotic ribosome biogenesis and function. RNA Biol 14, 1138-1152 (2016)). In addition, cationic lipids aid the delivery of nucleic acids, which are anionic, because they form complexes (See e.g., McIntyre, W. et al. Positive-sense RNA viruses reveal the complexity and dynamics of the cellular and viral epitranscriptomes during infection. Nucleic Acids Res 46, 5776-5791 (2018)).

However, the inventors have observed that if a composition or drug has a positive net charge, use of cationic lipids may problematically decrease the ability of the composition or drug to be loaded within liposomes. Another problematic issue is that cationic lipids on the outside of a vesicle are less likely to be compatible with delivery in vivo, as such vesicles will likely problematically stick non-specifically to the many anionic surfaces in a living organism, and can lead to undesirable phagocytosis (See e.g., Basanta-Sanchez, M., Temple, S., Ansari, S. A., D'Amico, A. & Agris, P. F. Attomole quantification and global profile of RNA modifications: Epitranscriptome of human neural stem cells. Nucleic Acids Res 44, e26 (2016); and Dunn, G., Boles, C. & Ventura, P. Complementary DNA Shearing and Size-selection Tools for Mate-pair Library Construction. J Biomol Tech 23, S36-S37 (2012)).

There is a continuing need for liposomes configured for safe and efficacious drug and/or biomolecule delivery that overcome the deficiencies of non-asymmetric lipid liposomes.

### SUMMARY

In embodiments, the present disclosure relates to one or more asymmetrical vesicles and use thereof such as for drug or biomolecule delivery, including stable asymmetric unilamellar vesicles. In embodiments, the drug or biomolecules have a positive or negative charge associated therewith and each may be in a pharmaceutically form, or a pharmaceutically acceptable salt form.

In embodiments, the present disclosure relates to a charged vesicle, including: a bilayer of lipids forming a shell, wherein the bilayer of lipids includes an inner layer of lipids and an outer layer of lipids, wherein the inner layer of lipids and the outer layer of lipids are different, and wherein the bilayer is characterized by having an asymmetric charge distribution; and an interior portion of the shell configured to entrap a drug or biomolecule.

In some embodiments, the present disclosure relates to a method for preparing a large unilamellar vesicle (LUV), including: contacting a cyclodextrin-lipid complex including one or more charged donor lipids and methyl-α-cyclodextrin with a liposome including a unilamellar membrane having an inner leaflet and an outer leaflet, to exchange one or more charged donor lipids from the cyclodextrin-lipid complex to the outer leaflet to form an asymmetrical large unilamellar vesicle.

In some embodiments, the present disclosure relates to a method for preparing a large unilamellar vesicle (LUV), including: contacting a cyclodextrin-lipid complex including one or more charged donor lipids and cyclodextrin with a liposome including a unilamellar membrane having an inner leaflet and an outer leaflet, to exchange one or more charged donor lipids from the cyclodextrin-lipid complex to the outer leaflet to form an asymmetrical large unilamellar vesicle.

In some embodiments, the present disclosure includes an asymmetrical large unilamellar vesicle made by a process including: contacting a cyclodextrin-lipid complex including one or more charged donor lipids and methyl-α-cyclodextrin with a liposome including a unilamellar membrane having an inner leaflet and an outer leaflet, to exchange one or more charged donor lipids from the cyclodextrin-lipid complex to the outer leaflet under conditions suitable to form an asymmetrical large unilamellar vesicle.

In some embodiments, the present disclosure includes an asymmetrical large unilamellar vesicle made by a process including: contacting a cyclodextrin-lipid complex including one or more charged donor lipids and cyclodextrin with a liposome including a unilamellar membrane having an inner leaflet and an outer leaflet, to exchange one or more charged donor lipids from the cyclodextrin-lipid complex to the outer leaflet under conditions suitable to form an asymmetrical large unilamellar vesicle.

In some embodiments, the present disclosure relates to a kit for substituting lipids in a unilamellar vesicle to form an asymmetric unilamellar vesicle including; at least one α-cyclodextrin; at least one first instruction for forming a cyclodextrin-lipid complex including the at least one lipid bound to the α-cyclodextrin; and at least one second instruction describing a method for using the at least one cyclodextrin-lipid complex to exchange the at least one lipid between a lipid bilayer of a liposome membrane and the cyclodextrin-lipid complex to form an asymmetric unilamellar vesicle.

In some embodiments, the present disclosure relates to a kit for substituting lipids in a unilamellar vesicle to form an asymmetric unilamellar vesicle including: at least one cyclodextrin; at least one first instruction for forming a cyclodextrin-lipid complex including the at least one lipid bound to the cyclodextrin; and at least one second instruction describing a method for using the at least one cyclodextrin-lipid complex to exchange the at least one lipid between a lipid bilayer of a liposome membrane and the cyclodextrin-lipid complex to form an asymmetric unilamellar vesicle.

The illustrative aspects of the present disclosure are designed to solve the problems herein described and/or other problems not discussed.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 depicts a schematic illustration of asymmetric LUVs preparation.

FIGS. 2A-2D depict a schematic illustration of TMA-DPH binding assay for measuring outer leaflet charge. FIG. 2A depicts the structure of TMA-DPH. FIGS. 2B, 2C, and 2D depict binding to net anionic, neutral and cationic outer leaflets, respectively. The signs show relative lipid charge.

FIG. 3 depicts the relationship between normalized fluorescence of TMADPH (F/F₀) and outer leaflet net charge in vesicles containing POePC and/or POPS as charged lipids. F/F₀ equals the fluorescence intensity of TMADPH in vesicle-containing samples/fluorescence of TMADPH in ethanol. Net Phospholipid Charge (mol %) equals the mol % net charge (of phospholipid) in the outer leaflet of asymmetric LUVs. Solid boxes: Mixed-charges sample: phospholipids were composed of 75 mol % of POPC and 25 mol % of POePC and POPS in various ratios. Open Boxes: Mono-charged sample: phospholipids were composed of various % POePC and POPC or of % POPS and POPC. The samples had 40 mol % cholesterol. Mean values and standard deviations from three vesicle preparations are shown. Experimental values for specific asymmetric LUVs preparations (arrows) are also shown. See Results in example section below for details. Mean values and standard deviations from three vesicle preparations are shown.

FIGS. 4A and 4B depict a comparison of TLC and TMADPH binding assay results for outer leaflet charge of asymmetric LUVs FIG. 4A depicts: Cationic/neutral outer leaflet vesicles: (a) POePC:POPC out/POPC in/Chol, (b) DOTAP:POPC out/POPC in/Chol, (c) POePC:POPC out/POPS:POPC in/Chol and (d) DOTAP:POPC out/POPG:POPC in/Chol, and FIG. 4B depicts: anionic outer leaflet vesicles: (e)

POPS:POPC out/POPC in/Chol, (f) POPG:POPC out/POPC in/Chol, (g) POPS:POPC out/POePC:POPC in/Chol and (h) POPG:POPC out/DOTAP:POPC in/Chol. Results show mean values and standard deviations from three vesicle preparations.

FIGS. 5A and 5B depict time dependence of outer leaflet charge assayed with TMA-DPH binding assay for asymmetric LUVs: FIG. 5A: POePC:POPC out/POPS:POPC in/Chol and FIG. 5B: POPS:POPC out/POePC:POPC in/Chol in PBS/sucrose or PBS. Results show mean values and standard deviations from three vesicle preparations.

FIG. 6 depicts dox entrapment within symmetric LUVs containing 40 mol % cholesterol and either 60 mol % POPC or 45mol % POPC and 15 mol % POePC, POPS or POPG. These samples were pelleted by centrifugation and washed twice to match the protocol used for asymmetric vesicles (see Methods below). Results show mean values and standard deviations from three vesicle preparations.

FIG. 7 depicts Dox entrapment within asymmetric LUVs. (a) POePC:POPC out/POPG:POPC in/Chol, (b) POePC:POPC out/POPS:POPC in/Chol, (c) POePC:POPC out/POPC in/Chol, (d) POPS:POPC out/POePC:POPC in/Chol, (e) POPG:POPC out/POePC:POPC in/Chol. Results show mean values and standard deviations from three vesicle preparations.

FIG. 8 depicts an example of TLC plate chromatographed in 3:1:1 (v:v) chloroform, methanol and acetic acid. Lane 1-5 standards: From left to right 2, 4, 8, 16 or 32 μg of POPC, POePC and POPS; Lane 6-9: POPS:POPC out/POPC in/Chol asymmetric LUVs; Lane 10-12: POPS:POPC out/POePC:POPC in/Chol asymmetric LUVs. Lanes 7, 9, and 11 are with donor having 25 mol % charged phospholipid. Lanes, 6, 8,10, and 12 had donor with 50 mol % charged phospholipid. In these cases, it was possible to achieve a reasonable lipid yield with 50 mol % charged phospholipid in donor, but the ability to use 50 mol % charged phospholipid is lipid-type dependent. Lanes 6 and 8, 7 and 9 and 10 and 12 are simple duplicate loadings.

FIG. 9 depicts a standard curve showing relationship between normalized fluorescence of TMADPH (F/F0) and outer leaflet net charge for asymmetric LUVs containing DOTAP and/or POPG. F/F0 equals the fluorescence intensity of TMADPH in vesicle-containing samples/fluorescence of TMADPH in ethanol. Net Phospholipid Charge (mol %) equals the mol % (of phospholipid) net charge in the outer leaflet of asymmetric LUVs. Solid boxes: mixed-charges samples: phospholipids were composed of 75 mol % of POPC and total of 25 mol % of DOTAP and POPG in various ratios. Open Boxes: Mono-charged samples: the phospholipids are composed of various % DOTAP and POPC or various % POPG and POPC. Results show mean values and standard deviations from three vesicle preparations.

FIG. 10 depicts DNA entrapment within symmetric LUVs containing 40 mol % cholesterol and either 60 mol % POPC or 45 mol % POPC and 15 mol % POePC, POPS or POPG. These samples were pelleted by centrifugation and washed twice to match the protocol used for asymmetric vesicles (see Methods below). Results show mean values and standard deviations from three vesicle preparations.

FIG. 11 is a plot depicting asymmetric vesicles with cationic POePC in their inner leaflets and cationic POPS or POPG in their inner leaflets (compositions a and b) trapped the largest amount of DNA, in amounts per lipid similar level to those in symmetric vesicles containing anionic lipids in both leaflets even though they have 2 times difference.

FIGS. 12A-12C depict a cartoon of a non-limiting example of a symmetric lipid vesicle (FIG. 12A) adjacent a non-limiting example of an asymmetric lipid vesicle (FIG. 12B). FIG. 12 C depicts various lipid components of the vesicles.

FIG. 13 depicts a protocol for preparing asymmetric vesicles with positive charge on one side of the membrane and negative charge on the other.

FIG. 14 depicts doxorubicin, a non-limiting example of a positively charged anticancer drug suitable for use in embodiments of the present disclosure.

FIG. 15 depicts a cartoon of expected loading ability of different charged liposomes towards doxorubicin.

FIGS. 16A and 16B depict dox trapped at highest levels when lipid on inside of vesicle has a minus charge.

FIG. 17 depicts loading of charged liposomes with nucleic acid is aided by positively charged lipids.

FIG. 18 depicts an embodiment where DNA entrapment is greatest when vesicles have a positively charged lipid on the inside.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

In embodiments, the present disclosure relates to one or more asymmetrical vesicles and use thereof such as for drug or biomolecule delivery. In embodiments, the drug or biomolecules have a positive or negative charge associated therewith. In embodiments, the drug or biomolecules may be pharmaceutically acceptable or in a a pharmaceutically acceptable salt form.

Further, the present disclosure is directed to a charged vesicle, including: a bilayer of lipids forming a shell, wherein the bilayer of lipids includes an inner layer of lipids and an outer layer of lipids, wherein the inner layer of lipids and the outer layer of lipids are different, and wherein the bilayer is characterized by having an asymmetric charge distribution; and an interior portion of the shell configured to entrap a drug. Advantages provided by embodiments of the present disclosure include enhanced drug delivery including the ability to trap many types of drugs such as charged drugs within a lipid bilayer or aqueous lumen of the charged vesicles of the present disclosure, easy manufacturing procedures to maintain drug bioactivity, high loading of drug to minimize the dosage needed, the ability to use multi-dosing to maintain an effective drug concentration, the ability to target specific cells, low leakage of drug from the vesicle after loading, and biocompatibility with long circulation times.

In embodiments, the present disclosure relates to the development and use of lipid-bound cyclodextrins to efficiently exchange lipids present in the outer leaflet of a liposome bilayer. In embodiments, charged exogenous lipids are bound to cyclodextrin molecules to form cyclodextrin-lipid complexes capable of exchanging the charged exogenous lipids bound thereto with endogenous lipids located in the outer leaflet of a liposome to form a stable asymmetric charged vesicle suitable for drug or biomolecule delivery. In embodiments, a stable asymmetric charged vesicle is provided in a pharmaceutically acceptable form.

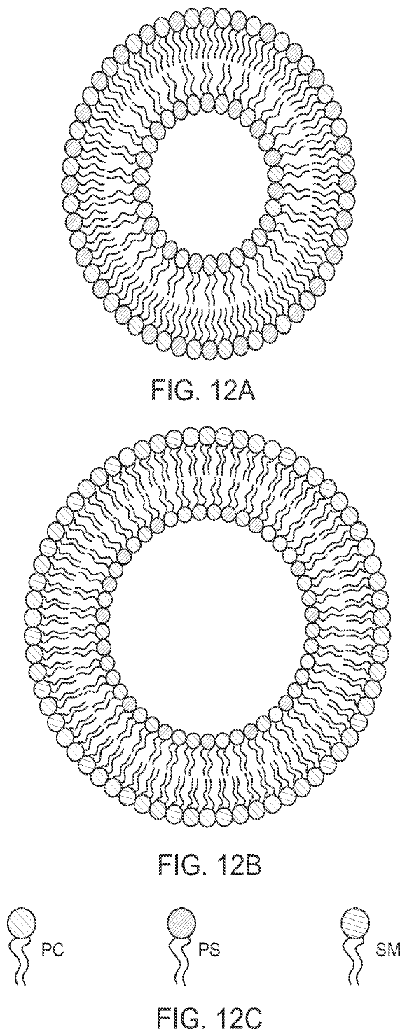

Referring now to FIG. 12, liposomal symmetry and asymmetry is depicted, wherein symmetric vesicles have the same lipids on each side of a membrane, and asymmetric vesicles have different lipids on each side of the membrane.

In some embodiments, the present disclosure relates to a method for preparing a large unilamellar vesicle (LUV), including: contacting a cyclodextrin-lipid complex including one or more charged donor lipids and methyl-α-cyclodextrin with a liposome including a unilamellar membrane having an inner leaflet and an outer leaflet, to exchange one or more charged donor lipids from the cyclodextrin-lipid complex to the outer leaflet to form an asymmetrical large unilamellar vesicle.

In some embodiments, the present disclosure relates to a method for preparing a large unilamellar vesicle (LUV), including: contacting a cyclodextrin-lipid complex including one or more charged donor lipids and cyclodextrin with a liposome including a unilamellar membrane having an inner leaflet and an outer leaflet, to exchange one or more charged donor lipids from the cyclodextrin-lipid complex to the outer leaflet to form an asymmetrical large unilamellar vesicle.

In some embodiments, the present disclosure includes an asymmetrical large unilamellar vesicle made by a process including: contacting a cyclodextrin-lipid complex including one or more charged donor lipids and methyl-α-cyclodextrin with a liposome including a unilamellar membrane having an inner leaflet and an outer leaflet, to exchange one or more charged donor lipids from the cyclodextrin-lipid complex to the outer leaflet under conditions suitable to form an asymmetrical large unilamellar vesicle.

In some embodiments, the present disclosure includes an asymmetrical large unilamellar vesicle made by a process including: contacting a cyclodextrin-lipid complex including one or more charged donor lipids and cyclodextrin with a liposome including a unilamellar membrane having an inner leaflet and an outer leaflet, to exchange one or more charged donor lipids from the cyclodextrin-lipid complex to the outer leaflet under conditions suitable to form an asymmetrical large unilamellar vesicle.

### Definitions

As used in the present specification, the following words and phrases are generally intended to have the meanings as set forth below, except to the extent that the context in which they are used

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [Cl 95%] for the mean) or within ±10% of the indicated value, whichever is greater.

As used herein, the term “biomolecule” refers to any type of organic molecule normally found in a living organism. Exemplary biomolecules include, but are not limited to, peptides, oligopeptides, lipids, nucleic acids, oligonucleotides, and carbohydrates. In one embodiment, a biomolecule is a single or double stranded nucleic acid, including single or double stranded DNA, RNA, or an isolated nucleic acid molecule. In embodiments, biomolecules of the present disclosure may have a charge. In embodiments, the term biomolecule includes a wild-type, synthetic, or recombinantly made biomolecule.

The term “binding”, “to bind”, “binds, “bound” or any derivation thereof refers to any direct interaction, e.g., chemical bond, between two or more molecules, including, but not limited to, covalent bonding, ionic bonding, hydrogen bonding. In some embodiments, the term extends to a hydrophobic effect where water molecules push hydrophobic molecules together. Thus, this term encompasses the interaction between a cyclodextrin and a lipid. More specifically, the interaction between the hydrophobic core of a cyclodextrin and a lipid, e.g., sphingolipid and/or phospholipid.

As used herein the term “cyclodextrin” or “CD” as used herein refers to a family of cyclic oligosaccharides, composed of five or more α-D-glucopyranoside units. In embodiments, cyclodextrins (CDs) are cyclic oligomers of glucose having, for example, six (α-cyclodextrins, α-CDs), seven β-cyclodextrins, β-CDs), or eight (γ-cyclodextrins, γ-CDs) glucose units. Cylclodextrins include a hydrophobic interior portion (cavity) capable of binding hydrophobic molecules. In embodiments, cyclodextrins of the present disclosure include a lipophilic central cavity and a hydrophilic outer surface. Non-limiting examples of cyclodextrins which can be incorporated in the cyclodextrin-lipid complexes of the present disclosure include, but are not limited, α-cyclodextrins. Non-limiting examples of α-cyclodextrins include methyl-α-cyclodextrins (e.g., a species of α-cyclodextrins with a methyl group or methyl groups attached to the glucose rings of a cyclodextrin, such as dimethyl-α-cyclodextrin and randomly methylated alpha cyclodextrins), sulfo-α-cyclodextrin, and hydroxypropyl-α-cyclodextrin, carboxyethyl-α-cyclodextrin, succinyl-α-cyclodextrin, hydroxyethyl-α-cyclodextrin, ethyl-α-cyclodextrin, and n-butyl-α-cyclodextrin. In embodiments, cyclodextrins which can be incorporated in the cyclodextrin-lipid complexes of the present disclosure include, but are not limited, α-cyclodextrins, β-cyclodextrins, γ-cyclodextrins, or combinations thereof.

The term “cyclodextrin-lipid complex” or “CD-lipid complex” as used herein refers to a complex that is formed between a lipid and at least one cyclodextrin where the lipid or lipids are bound to the cyclodextrin(s) (at their hydrophobic interior cavity). In some embodiments, a cyclodextrin-lipid complex includes a plurality of cyclodextrin molecules bound to a lipid. In embodiments, a cyclodextrin-lipid complex of the present disclosure includes a lipid bound to a single cyclodextrin molecule. In some embodiment, a ratio of cyclodextrin to lipid may be as high as 4:1. In embodiments, two cyclodextrins may be bound to two acyl chains.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position.

As used herein the terms “drug,” “drug substance,” “active pharmaceutical ingredient,” and the like, refer to a compound (e.g., doxorubicin or dox) that may be used for treating a subject in need thereof.

An “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

As used herein the term “pharmaceutically acceptable vehicle” refers to a diluent, adjuvant, excipient or carrier with which a compound such as a drug is administered.

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated; however, the resulting reaction product can be produced directly from a reaction between the added reagents or from an intermediate from one or more of the added reagents which can be produced in the reaction mixture.

“Conversion” and “converting” refer to a process including one or more steps wherein a species is transformed into a distinct product.

The term “lipid” or “lipids” used herein refers to an organic molecule that is insoluble in water and soluble in non-polar solvents. Lipids include fatty acids, esters derived from a fatty acid and a long-chain alcohol, triacylglycerol, phospholipids, prostaglandin, sphingolipids, and sterols. Lipids of the present disclosure can be, for example, labeled, such as lipids labeled with a fluorescent dye, or incorporate a radioactive isotope (e.g., ¹⁴C or ³H). Lipids can be a naturally occurring lipid that has been created synthetically or isolated from cells. In some embodiments, the lipids of the present disclosure can be an “unnatural lipid”, or a lipid that is not found in nature.

Unnatural lipids include, for example, lipids with a modified acyl chain, length(s), composition, function or a combination thereof when compared to its naturally occurring (unmodified) counterpart, such as, for example, N-hepadecanoyl-D-erythro-sphingosylphosphorylcholine (C₁₇:0 SM). In some instances, unnatural lipids include lipid analogs that are modified in such a manner that they are not subject to phospholipase mediated enzymatic activity.

As used herein the term “pharmaceutically acceptable” substances refers to those substances, which are within the scope of sound medical judgment suitable for use in contact with the tissues of subjects without undue toxicity, irritation, allergic response, and the like, and effective for their intended use.

As used herein the term “pharmaceutical composition” refers to the combination of one or more drug substances or biomolecules and one or more vesicles of the present disclosure suitable for administration to a subject.

As used herein, the term “pharmaceutically acceptable salt” refers to a salt of a compound, which possesses the desired pharmacological activity of the parent compound. Non-limiting examples of pharmaceutically acceptable salts include: acid addition salts, formed with inorganic acids such as hydrochloric acid, hydrobromic acid, sulfuric acid, nitric acid, phosphoric acid, and the like; or formed with organic acids; and salts formed when an acidic proton present in the parent compound is replaced by a metal ion, for example, an alkali metal ion, an alkaline earth ion, or an aluminum ion. In embodiments, drugs or biomolecules of the present disclosure may be in a pharmaceutically acceptable salt form.

“Phospholipids” as used herein means a class of lipids that contain a phosphate group attached to two fatty acid chains by a glycerol molecule. The phosphate group typically forms a negatively charged polar head, which is hydrophilic. In certain embodiments, the net charge of a lipid can be neutral when the polar group attached to the phosphate group by a phosphoester is positively charged a positive charge. In embodiments, an ethyl group may attach the phosphate and neutralize the charge and result in a lipid with a positive charge. In embodiments, the fatty acid chains form uncharged, non-polar tails, which are hydrophobic. Non-limiting examples of phospholipids of the present disclosure are those present in the outer leaflet of the cell membrane, such as phosphatidylcholine (PC). Phosphatidylcholines likely to be in the outer leaflet include 1-dioleoyl phosphatidylcholine (DOPC), 1-palmitoyl 2-oleoyl phosphatidylcholine (POPC) and 1-stearoyl-2-oleoyl-phosphatidylcholine (SOPC). In embodiments, phospholipid includes artificial lipid such as POePC. Additional PCs that are present in membranes would be analogous to those above, but with linoleic acid, linolenic acid, arachidonic acid or docosahexenoic acid in the 2 position. In certain embodiments, these latter species can be found in the inner leaflet, but in the absence of methods that can accurately analyze lipid asymmetry.

The term “subject” as used herein refers to any individual or patient to which the subject methods are performed. Generally the subject is human, although as will be appreciated by those in the art, the subject may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. In addition, the term “subject” may refer to a culture of cells, where the methods of the invention are performed in vitro to assess, for example, efficacy of a therapeutic agent.

Before embodiments are further described, it is to be understood that this disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DETAILED DESCRIPTION OF CERTAIN EMBODIMENTS

Lipid vesicles (liposomes) are artificial membranes that contain an aqueous interior surrounded by a bilayered shell of lipids, the oils the form biomembranes. Embodiments of the present disclosure described herein provide, inter alia, methods to change the lipids in the outer layer of vesicles so that one type or types of lipid face the external solution, and another type or types of lipid face the aqueous interior (e.g. asymmetric vesicles—See e.g. FIG. 12B) of the vesicle.

In embodiments, the methods of the present disclosure provide liposomes that have an asymmetric charge distribution, e.g. with different net charge on the lipids in the inner and outer layers. In embodiments, the inside or inner layer and outside or outer layer charge range could be any of 0-25% of negative charge inside with any of 0-25% of positive charge outside or any of 0-25% of positive charge inside with any of 0-25% of negative charge outside.

In embodiments, the inside or inner layer and outside or outer layer charge range could be any of 1-25% of negative charge inside with any of 1-25% of positive charge outside or any of 1-25% of positive charge inside with any of 1-25% of negative charge outside. For example, in some embodiments, liposomes include an outside which is negatively charged and an inside positively charged, as well as liposomes in which the outside is positively charged, and the inside is negatively charged. In embodiments, an entrapment of one or more charged drugs or biomolecules disposed within the vesicle may be controlled by the charge on the inner lipid layer, so e.g., that the entrapment of a positively charged drug increases and its leakage out of the vesicles slowed when the vesicles had a negatively charged inner layer. This is largely independent of the outer layer lipid composition.

In embodiments, it is possible to choose inner layer lipids to maximize liposomal loading of charged drugs or charged biomolecules independently of the identity of outer layer lipids. In embodiments, it is possible to independently vary outer layer lipids to, for example, impart favorable bioavailability and/or biodistribution properties. In embodiments, it is possible to use the same procedures to make improved liposomes for trapping anionic molecules to deliver to cells, including DNA, RNAs, or predetermined or previously isolated nucleic acid molecules.

In embodiments, lipid vesicles (liposomes) of the present disclosure include artificial membranes that contain an aqueous interior surrounded by a bilayered shell comprising or consisting of lipids, which are the molecules that form the essential portion of biomembranes. In embodiments, the present disclosure relates to one or more synthetic charged vesicles, including: a bilayer of lipids forming a shell, wherein the bilayer of lipids includes an inner layer of lipids and an outer layer of lipids, wherein the inner layer of lipids and the outer layer of lipids are different, and wherein the bilayer is characterized by having an asymmetric charge distribution; and an interior portion of the shell having a void suitable for holding a composition such as a drug or biomolecule. In embodiments, the asymmetric charge distribution is predetermined depending upon the charge of the cargo (e.g., drug or biomolecule). In embodiments, each layer of lipids is continuous in a spherical or round shape.

In some embodiments, a method to prepare lipid vesicles with lipids is provided so as to have an asymmetric charge distribution, e.g. with different net charge on the lipids in the inner and outer layers. For example, liposomes in which the outside is anionic and the inside cationic, as well as liposomes in which the outside is cationic and the inside is anionic. In embodiments, liposomes in which the outside has a predetermined charge and the inside has a predetermined charge different than the outside, such as liposomes in which the outside is cationic and the inside is anionic. In embodiments, methods of the present disclosure provide asymmetric vesicles in which the outer layers are cationic or anionic and the inner layer lipids are neutral. In some embodiments, the method can be used to prepare large unilamellar vesicles for drug delivery or biomolecule, with up to 25-50% charged phospholipid in each layer. In some embodiments, the method can be used to prepare large unilamellar vesicles for drug or biomolecule delivery, with a preselected percentage (such as mol %) of charged phospholipid in each layer such as a percentage up to 25%, up to 50%, up to 60%, up to 70%, or between 5% and 50%. In embodiments, the inclusion of a high amount of cholesterol provides a reproducible good yield of asymmetric liposomes.

Embodiments of the present disclosure may be used with various lipids. For example, in some embodiments, mixtures containing uncharged lipids including cholesterol and a zwitterionic lipid (phosphatidycholine), one of two different cationic lipids (0-ethyl phosphatidyl choline or dioleoyl-3-trimethylammoniurn propane) and one or three different anionic lipids (phosphatidylglycerol, phosphatidylserine or phosphatidic acid) can be used.

In embodiments, the vesicle constituents are provided in a predetermined amount. For example, cholesterol may be provided at 20% to 50%. In embodiments, neutral lipids, such as in a phospholipid ratio, may be provided at 50% to 100%. In embodiments, the charged lipids in a phospholipid ratio could be 0% to 50%, such as 1% to 50%. In embodiments, charged lipids may be provided in any percentage or ratio. In embodiments, percentage refers to mol(e) percentage. In embodiments, mole percent is the percentage of the total moles compound.

As described below, a comparison of the behavior of symmetric and asymmetric vesicles demonstrates a level of entrapment of a charged drug, e.g., the cationic drug doxorubicin, controlled by the charge on the inner lipid layer such that entrapment could be increased, and leakage of the vesicles slowed when the vesicles had an anionic inner layer. This was largely independent of the outer layer lipid composition. With embodiments of the present disclosure, it is possible to choose inner layer lipids to maximize liposomal loading of charged drugs or charged biomolecules independently of the identity of outer layer lipids. This result indicates that the method can independently vary outer layer lipids to, for example, impart favorable bioavailability and biodistribution properties. The methods also provide improved liposomes for trapping anionic molecules to deliver to cells or a subject in need thereof, including DNA and RNAs, or predetermined or previously isolated nucleic acid molecules or oligonucleotides.

In a further embodiment, the methods of the present disclosure provide liposomal vesicles that contain various drugs or biomolecules at higher levels than at present and results in more efficient uptake by cells. This includes anticancer drugs such as doxorubicin, or nucleic acids such as including DNAs and RNAs that code for antigens. This also includes DNAs and/or RNAs included in a method to provoke an immune reaction, e.g. to SARS-CoV-2 virus that causes COVID-19. Additional examples of nucleic acids include those found in one or more of the three vaccine types currently approved for use in the United States for SARS-Cov-2 and variants thereof such as: Pfizer-BioNTech (See e.g., Polack, F. P., Thomas, S. J., Kitchin, N., et al., 2020. N Engl J Med 383(27), 2603-2615), Moderna (See e.g., Baden, L. R., El Sahly, H. M., Essink, B., et al., 2021. N Engl J Med 384(5), 403-416) and Johnson & Johnson (See e.g., Sadoff, J., Gray, G., Vandebosch, A., et al., 2021. N Engl J Med 384, 2187-2201) including nucleotide-modified RNA (modRNA) encoding the SARS-CoV-2 full-length spike protein, modified by two proline alterations, mRNA-based vaccine that encodes a prefusion stabilized full-length spike protein of the severe acute respiratory syndrome coronavirus 2, or a recombinant, replication-incompetent human adenovirus type 26 (Ad26) vector encoding a full-length, membrane-bound SARS-CoV-2 spike protein. Other drugs, such as charged drugs, and other biomolecules, such as charged biomolecules, known in the art are suitable for use herein. In embodiments charge may refer to a net positive charge or a net negative charge.

In a further embodiment, vesicles containing drugs or biomolecules trapped in asymmetric liposomes with opposite net charges in their inner and outer leaflets may be provided for the purpose of maximizing drug loading within the interior of the liposome and controlling the amount of drug or biomolecule that can be delivered to cells. In embodiments, drugs or biomolecules trapped in asymmetric liposomes may be administered to a subject in need thereof.

In embodiments, the drug and/or biomolecule trapped in asymmetric liposomes of the present disclosure are provided in an amount sufficient to alter the subject, and/or have a beneficial therapeutic effect upon the subject.

In some embodiments, the drug and/or biomolecule trapped in asymmetric liposomes of the present disclosure are suitable for administering to a subject in need thereof. For example, a subject afflicted by disease or injury and in need of a treatment may be administered a drug and/or biomolecule trapped in asymmetric liposomes of the present disclosure including the one or more drugs or biomolecules relating to the treatment. In embodiments, the drug and/or biomolecule trapped in asymmetric liposomes of the present disclosure are applied to a site of disease or injury in a subject in need thereof. In embodiments, a subject in need thereof is a human or non-human mammal. In embodiments, one or more drugs and/or biomolecules trapped in one or more asymmetric liposomes of the present disclosure are provided in an amount effective to treat a subject in need thereof. In embodiments, a drug and/or biomolecule trapped in asymmetric liposomes of the present disclosure, or compositions including a drug and/or biomolecule trapped in asymmetric liposomes of the present disclosure may be administered to a subject in need thereof in a therapeutically effective amount or an amount that, when administered to a subject for treating or preventing a disease or illness, is sufficient to effect such treatment or prevention of disease or illness and related symptoms. A “therapeutically effective amount” can vary depending, for example, on the compound, the severity of the infection, the etiology of the infection, the age of the subject to be treated, comorbidities of the subject to be treated, existing health conditions of the subject, and/or the weight of the subject to be treated. In embodiments, a “therapeutically effective amount” is an amount sufficient to alter the subjects' natural state. As used herein the term “treat”, “treating” and “treatment” of disease or illness means an intervention for reducing the frequency of symptoms of a disease or illness, eliminating the symptoms of a disease or illness, avoiding or arresting the development of symptoms of a disease or illness, ameliorating or curing an existing or undesirable symptom caused by a disease or illness, and/or reducing the severity of symptoms of a disease or illness.

### Compositions

In embodiments, the present disclosure includes the formation of cyclodextrin-lipid complexes for use in the efficient exchange of lipids in liposomes such as acceptor liposomes. Generally, the cyclodextrin-lipid compositions of the present disclosure are formed by mixing phospholipids and/or in a solvent (e.g., an organic solvent). In embodiments, the lipids may then be dried to remove the solvent (e.g., nitrogen or vacuum). In embodiments, the dried lipids are mixed with an aqueous buffer, such as PBS or medium, to form multilamellar vesicles (MLV). The mixture of cyclodextrin and MLV are then incubated together to form cyclodextrin-lipid complexes. In embodiments, during incubation, lipids may separate from the MLV and bind to the hydrophobic interior cavity of a cyclodextrin molecule to form a cyclodextrin-lipid complex. In embodiments, certain cyclodextrins, such as α-cyclodextrins, have a unique hydrophobic cavity that is too small to bind cholesterol.

In some embodiments, cyclodextrin-lipid complexes of the present disclosure include α-cyclodextrin. In some specific embodiments, the alpha-cyclodextrin is a dimethyl-α-cyclodextrin, sulfo-α-cyclodextrin, and hydroxypropyl-α-cyclodextrin, carboxyethyl-α-cyclodextrin, succinyl-α-cyclodextrin, hydroxyethyl-α-cyclodextrin, ethyl-α-cyclodextrin, or n-butyl-α-cyclodextrin. In yet another embodiment, the cyclodextrin is hydroxypropyl-α-cyclodextrin. In embodiments, the cyclodextrin used to form a cyclodextrin-lipid complex of the present disclosure is methyl-α-cyclodextrin.

In some embodiments, cyclodextrin-lipid complexes of the present disclosure include α-cyclodextrin. In embodiments, cyclodextrins which can be incorporated in the cyclodextrin-lipid complexes of the present disclosure include, but are not limited, α-cyclodextrins, β-cyclodextrins, γ-cyclodextrins, or combinations thereof. In some embodiments, all different cyclodextrins can be used, either alpha, beta or gamma cyclodextrin, or alpha, beta or gamma cyclodextrin, modified with methyl, hydroxyl or other functional groups.

In embodiments, the lipids bound to cyclodextrin are lipids commonly found in the cell membrane such as, for example, lipids of the outer leaflet of the plasma membrane. For example, any lipid that includes a polar head group and acyl chain(s) can be used to form cyclodextrin-lipid complexes of the present disclosure. In specific embodiments, the lipids are exogenous phospholipids. In some embodiments of the present disclosure, the phospholipids is phosphatidylcholine (PC) or a derivative thereof. Non-limiting examples of suitable lipids for use herein include 1-palmitoyl-2-oleoyl-sn-glycero-3-phosphocholine (POPC), 1-palmitoyl-2-oleoyl-sn-glycero-3-ethylphosphocholine (chloride salt) (POePC), 1,2-dioleoyl-3-trimethylammonium-propane (chloride salt) (DOTAP), 1-palmitoyl-2-oleoyl-sn-glycero-3-phospho-(1′-rac-glycerol) (sodium salt) (POPG), 1-palmitoyl-2-oleoyl-sn-glycero-3-phospho-L-serine (sodium salt) (POPS), 1-palmitoyl-2-oleoyl-sn-glycero-3-phosphate (sodium salt) (POPA), and combinations thereof.

In some embodiments, the present disclosure relates to a charged vesicle, such as a stable charged vesicle. In embodiments, a charged vesicle includes a bilayer of lipids forming a shell, wherein the bilayer of lipids is characterized by having an asymmetric charge distribution; and an interior portion of the shell configured to entrap a drug (such as a positively charged drug or pharmaceutically acceptable salt thereof), or biomolecule (such as a negatively charged DNA or RNA or pharmaceutically acceptable salt thereof).

In embodiments, a charged vesicle includes a bilayer of lipids forming a shell, wherein the bilayer of lipids includes an inner layer of lipids and an outer layer of lipids, wherein the inner layer of lipids and the outer layer of lipids are different, and wherein the bilayer is characterized by having an asymmetric charge distribution; and an interior portion of the shell configured to entrap a drug or biomolecule. In some embodiments, the inner layer of lipids has a first net charge and the outer layer of lipids have a second net charge different than the first net charge. In some embodiments, the first net charge is positive, and the second net charge is negative. In some embodiments, the first net charge is negative, and the second net charge is positive. In some embodiments, the drug has a positive or negative charge. In some embodiments, the inner layer is negative, and the drug is positive, and a leakage of the drug is reduced compared to a non-charged vesicle including the same drug. In some embodiments, the inner layer is positive, and the drug or biomolecule is negative, and a leakage of the drug or biomolecule is reduced compared to a non-charged vesicle including the same drug or biomolecule. In some embodiments, the interior portion includes an aqueous medium. In some embodiments, the drug is a negatively charged DNA or RNA. In some embodiments, the inner layer of lipids has a neutral charge and the second net charge is positive or negative. In some embodiments, the inner layer of lipids and outer layer of lipids each include charged phospholipids in the amount of 25-50% of each layer of lipids. In some embodiments, the inner layer and the outer layer further include cholesterol. In some embodiments, the inner layer of lipids and outer layer of lipids include a mixture one or more uncharged lipids, one or more cationic lipids, one or more anionic lipids. In some embodiments, the inner layer of lipids and the outer layer of lipids include a mixture of two uncharged lipids, two cationic lipids, and three anionic lipids. In some embodiments, the two uncharged lipids are cholesterol and zwitterionic lipid. In some embodiments, the zwitterionic lipid is phosphatidylcholine (Popc). In some embodiments, the two cationic lipids comprise O-ethyl phosphatidyl choline or dioleoyl-3-trimethylammonium propane. In some embodiments, the three anionic lipids include phosphatidylglycerol, phosphatidylserine, and phosphatidic acid. In embodiments, the amount of anionic lipid, zwitterionic lipid, and cationic lipid are preselected and provided in preselected ratios to form an asymmetric charged vesicle in accordance with the present disclosure.

In embodiments, a charged vesicle includes a bilayer of lipids forming a shell, wherein the bilayer of lipids includes an inner layer of lipids and an outer layer of lipids, wherein the inner layer of lipids and the outer layer of lipids are different, and wherein the bilayer is characterized by having an asymmetric charge distribution; and an interior portion of the shell configured to entrap a drug or biomolecule. In some embodiments, the inner layer of lipids has a first net charge, and the outer layer of lipids have a second net charge different than the first net charge. In some embodiments, the first net charge is positive, and the second net charge is negative. In some embodiments, the first net charge is negative, and the second net charge is positive. In some embodiments, the drug has a positive or negative charge. In some embodiments, the inner layer is negative, and the drug is positive, and a leakage of the drug is reduced compared to a non-charged vesicle including the same drug. In some embodiments, the inner layer is positive, and the drug or biomolecule is negative, and a leakage of the drug or biomolecule is reduced compared to a non-charged vesicle including the same drug or biomolecule. In some embodiments, the interior portion includes an aqueous medium.

In embodiments, a charged vesicle includes a bilayer of lipids forming a shell, wherein the bilayer of lipids includes an inner layer of lipids and an outer layer of lipids, wherein the inner layer of lipids and the outer layer of lipids are different, and wherein the bilayer is characterized by having an asymmetric charge distribution; and an interior portion of the shell configured to entrap a drug or biomolecule. In some embodiments, the inner layer of lipids has a first net charge, and the outer layer of lipids have a second net charge different than the first net charge. In some embodiments, the first net charge is positive, and the second net charge is negative. In some embodiments, the first net charge is negative, and the second net charge is positive. In some embodiments, the drug has a positive or negative charge. In some embodiments, the drug is a negatively charged DNA or RNA. In some embodiments, the inner layer of lipids has a neutral charge and the second net charge is positive or negative.

In embodiments, a charged vesicle includes a bilayer of lipids forming a shell, wherein the bilayer of lipids includes an inner layer of lipids and an outer layer of lipids, wherein the inner layer of lipids and the outer layer of lipids are different, and wherein the bilayer is characterized by having an asymmetric charge distribution; and an interior portion of the shell configured to entrap a drug or biomolecule. In some embodiments, the inner layer of lipids has a first net charge, and the outer layer of lipids have a second net charge different than the first net charge. In some embodiments, the first net charge is positive, and the second net charge is negative. In some embodiments, the first net charge is negative, and the second net charge is positive. In some embodiments, the drug has a positive or negative charge. In some embodiments, the inner layer is negative, and the drug is positive, and a leakage of the drug is reduced compared to a non-charged vesicle including the same drug. In some embodiments, the inner layer of lipids and outer layer of lipids each include charged phospholipids in the amount of 25-50% of each layer of lipids. In some embodiments, the inner layer and the outer layer further include cholesterol. In some embodiments, the inner layer of lipids and outer layer of lipids include a mixture one or more uncharged lipids, one or more cationic lipids, one or more anionic lipids. In some embodiments, the inner layer of lipids and the outer layer of lipids include a mixture of two uncharged lipids, two cationic lipids, and three anionic lipids. In some embodiments, the two uncharged lipids are cholesterol and zwitterionic lipid.

### Methods

In embodiments, the methods of the present disclosure provide a lipid exchange process by which a lipid is bound to a cyclodextrin to form a cyclodextrin-lipid composition (i.e., cyclodextrin-lipid complex). Cyclodextrin-lipid complexes are then incubated with liposomes under certain conditions in order to facilitate the efficient exchange of the lipids bound to the cyclodextrin-lipid complexes and the endogenous to membrane lipids located within the liposome membrane.

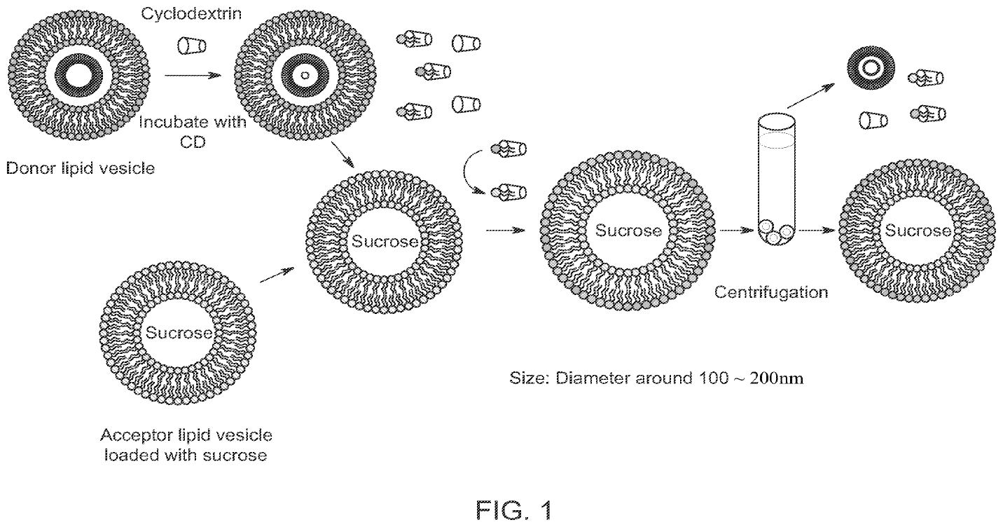

In embodiments, the lipid exchange methods of the present disclosure generally include the formation and use of cyclodextrin-lipid complexes, as described above or shown in FIG. 1. More specifically, the present methods include the formation and use of cyclodextrin-lipid complexes composed of an alpha-cyclodextrin and a lipid. In embodiments, the lipid-exchange methods of the present disclosure include the formation and use of cyclodextrin-lipid complexes composed of a methyl-alpha-cyclodextrin and a lipid.

In embodiments, the cyclodextrins are an alpha-cyclodextrin. As noted above, α-cyclodextrins have a unique structure that provides a unique capability to bind certain lipids, but not sterols (i.e., cholesterol). Specifically, α-cyclodextrins have a smaller hydrophobic cavity compared to other classes of cyclodextrin, such as β-cyclodextrin and γ-cyclodextrin, which prohibits sterol binding, and thus cell death. In certain embodiments, the alpha-cyclodextrin is a dimethyl-α-cyclodextrin, sulfo-α-cyclodextrin, and hydroxypropyl-α-cyclodextrin, carboxyethyl-α-cyclodextrin, succinyl-α-cyclodextrin, hydroxyethyl-α-cyclodextrin, ethyl-α-cyclodextrin, and n-butyl-α-cyclodextrin.

In embodiments, the cyclodextrin used to form a cyclodextrin-lipid complex of the present disclosure is methyl-α-cyclodextrin. In yet another embodiment, the cyclodextrin is hydroxypropyl-α-cyclodextrin.

In embodiments, cyclodextrins which can be incorporated in the cyclodextrin-lipid complexes of the present disclosure include, but are not limited, α-cyclodextrins, β-cyclodextrins, γ-cyclodextrins, or combinations thereof. In some embodiments, all different cyclodextrins can be used, either alpha, beta or gamma cyclodextrin, or alpha, beta or gamma cyclodextrin, modified with methyl, hydroxyl or other functional groups.

In certain embodiments of the present disclosure, the lipids incorporated in cyclodextrin-lipid complexes are lipids commonly found in the outer leaflet of a liposome membrane. For example, any lipid that includes a polar head group and acyl chain(s) can be used to form cyclodextrin-lipid complexes of the present disclosure. In specific embodiments, the lipids used for exchange are phospholipids or sphingolipids.

In some embodiments of the present disclosure, the sphingolipid is a sphingomyelin or a derivative thereof. In specific embodiments of the present disclosure, the phospholipid is phosphatidylcholine or a derivative thereof. In yet another embodiment, the cyclodextrin-lipid complex includes sphingomyelin (SM), 1-dioleoyl phosphatidylcholine (DOPC), 1-palmitoyl 2-oleoyl phosphatidylcholine (POPC), 1-stearoyl-2-oleoyl-phosphatidylcholine (SOPC) and/or combinations thereof. In yet another embodiment, the cyclodextrin-lipid complex is preselected to include a charged cationic lipid, anionic lipid, or combinations thereof. In yet another embodiment, the cyclodextrin-lipid complex is preselected to include a charged cationic lipid.

In some embodiments, the present disclosure relates to a method for preparing a large unilamellar vesicle (LUV), including: contacting a cyclodextrin-lipid complex including one or more charged donor lipids and methyl-α-cyclodextrin with a liposome including a unilamellar membrane having an inner leaflet and an outer leaflet, to exchange one or more charged donor lipids from the cyclodextrin-lipid complex to the outer leaflet to form an asymmetrical large unilamellar vesicle. In some embodiments, the method includes forming a cyclodextrin-lipid complex with a preselected ratio of charged lipids to neutral lipids. Non-limiting examples of suitable ratios include a ratio of charged lipid to neutral lipid of 0:100, 1:99, 25:75, 50:50, 99:1, 100:0, or between 1:99 and 99:1. In some embodiments, the charged lipids include one or more of 1 palmitoyl-2-oleoyl-sn-glycero-3-ethylphosphocholine (POePC), 1,2-dioleoyl-3-triethylammonium-propane (chloride salt) (DoTAP), 1-palmitoyl-2-oleoyl-sn-glycero-3-phospho-L-serine (sodium salt) (POPS), 1-palmitoyl-2-oleoyl-sn-glycero-3-phospho-(1′-rac-glycerol)(sodium salt) (POPG), or 1-palmitoyl-2-oleoyl-sn-glycero-3-phosphate-L-serine (sodium salt) (POPA). In some embodiments, the charged lipids are selected from the group consisting of 1 palmitoyl-2-oleoyl-sn-glycero-3-ethylphosphocholine (POePC), 1,2-dioleoyl-3-triethylammonium-propane (chloride salt) (DoTAP), 1-palmitoyl-2-oleoyl-sn-glycero-3-phospho-L-serine (sodium salt) (POPS), 1-palmitoyl-2-oleoyl-sn-glycero-3-phospho-(1′-rac-glycerol)(sodium salt) (POPG), or 1-palmitoyl-2-oleoyl-sn-glycero-3-phosphate-L-serine (sodium salt) (POPA), and combinations thereof.

In some embodiments, the liposome includes a unilamellar membrane having an inner leaflet and an outer leaflet including a preselected ratio of charged lipids and cholesterol. Non-limiting examples of suitable ratios include a ratio of charged lipid to cholesterol of 0:100, 1:99, 25:75, 50:50, 99:1, 100:0, or between 1:99 and 99:1. In some embodiments, the charged lipids include one or more of 1 palmitoyl-2-oleoyl-sn-glycero-3-ethylphosphocholine (POePC), 1,2-dioleoyl-3-triethylammonium-propane (chloride salt) (DoTAP), 1-palmitoyl-2-oleoyl-sn-glycero-3-phospho-L-serine (sodium salt) (POPS), 1-palmitoyl-2-oleoyl-sn-glycero-3-phospho-(t-rac-glycerol)(sodium salt) (POPG), 1-palmitoyl-2-oleoyl-sn-glycero-3-phosphate-L-serine (sodium salt) (POPA).

In some embodiments, contacting further includes incubating the cyclodextrin-lipid complex and the liposome in a solution under conditions such that a plurality of lipids are exchanged between the cyclodextrin-lipid complex and the outer leaflet in an amount sufficient to provide a net charge to the outer leaflet opposite to the net charge of the inner leaflet. For example, in some embodiments, a new charge is provided to the outer leaflet opposite to the net charge of the inner leaflet. In some embodiments, the incubation occurs for a duration between 30 minutes and 2 hours.

In some embodiments, the lipid is an unnatural lipid or includes a label. In some embodiments, the label is selected from the group consisting of a fluorescent dye and a radioisotope.

In some embodiments, forming a multilamellar vesicle including at least one lipid prior to forming the cyclodextrin-lipid complex is provided. In embodiments, forming the cyclodextrin-lipid complex includes incubating said multilamellar vesicle with a solution including a cyclodextrin. In some embodiments, the incubation occurs at about 37° C. for about 30 minutes. In some embodiments, the cyclodextrin is not α-cyclodextrin or β-cyclodextrin.

In embodiments, the present disclosure includes preparing a donor lipid, such as a donor lipid loaded MαCD for lipid exchange. In embodiments, preselected ratios of charged lipids (such as e.g, POePC, DOTAP, POPS, or POPG) and zwitterionic POPC may be dissolved in a solvent such as chloroform and combined, dried, and then subjected to high vacuum. In embodiments, dried lipids may be hydrated such as by adding them to a water bath and dispersing at 70° C. with an aliquot of pre-warmed PBS, and then an aliquot of pre-warmed MαCD, to give a predetermined final concentration of MαCD and lipid, such as e.g., 40 mM MαCD and 16 mM lipid. The mixtures may be vortexed briefly, and then vortexed in a multitube vortexer for 2 h at 55° C., cooled to room temperature, covered in foil, and reserved for further use. In embodiments, neutral lipids may be provided in a phospholipid ratio of 50% to 100%. In embodiments, the balance includes charged lipid. In embodiments, the charged lipids in the phospholipid ratio could be 0% to 50%, or 1 mol % to 50 mol %. In embodiments, various charged lipids may be provided in any ratios.

In some embodiments, the present disclose includes preparation of acceptor LUV for lipid exchange. For example, preselected ratios of charged lipids (e.g., POePC, DOTAP, POPS, or POPG), zwitterionic POPC, and cholesterol (40 mol % of total lipid) may be dissolved in a solvent such as chloroform and combined. LUVs may be prepared as described above for symmetric vesicles. In embodiments, 15 cholesterol may be provided in a ratio or 20 mol % to 50 mol %, and the balance is phospholipid. In embodiments, the neutral lipids in the phospholipid ratio may be 75 mol % to 100 mol % (the balance will be charged lipid). In embodiments, the charged lipids in the phospholipid ratio may be 0% to 25 mol % or 1 mol % to 25 mol %. The different charged lipids could be in any ratios.

In embodiments, an outer leaflet lipid exchange is performed. In embodiments, lipid pellets may be suspended in an aqueous medium, and donor lipid-MαCD mixture and acceptor LUVs mixtures combined and mixed to form lipid-exchange mixtures. In embodiments, the asymmetric LUVs are formed and isolated and/or are substantially purified.

In embodiments, suitable methods for forming the asymmetrical charged vesicles of the present disclosure include drying lipids into film, and subsequently hydrating the dried lipids with aqueous solution. In embodiments, the methods include injecting alcohol (e.g., ethanol) dissolved lipid mixtures into aqueous solutions, using microfluidic devices and then dialyzing, or directly using dialysis to prepare liposomes. In some embodiments, the present disclosure relates to a method for preparing a large unilamellar vesicle (LUV), including: contacting a cyclodextrin-lipid complex including one or more charged donor lipids and methyl-α-cyclodextrin with a liposome including a unilamellar membrane having an inner leaflet and an outer leaflet, to exchange one or more charged donor lipids from the cyclodextrin-lipid complex to the outer leaflet to form an asymmetrical large unilamellar vesicle.

In some embodiments, the present disclosure relates to a method for preparing a large unilamellar vesicle (LUV), including: contacting a cyclodextrin-lipid complex including one or more charged donor lipids and cyclodextrin with a liposome including a unilamellar membrane having an inner leaflet and an outer leaflet, to exchange one or more charged donor lipids from the cyclodextrin-lipid complex to the outer leaflet to form an asymmetrical large unilamellar vesicle.

In some embodiments, the present disclosure includes an asymmetrical large unilamellar vesicle made by a process including: contacting a cyclodextrin-lipid complex including one or more charged donor lipids and methyl-α-cyclodextrin with a liposome including a unilamellar membrane having an inner leaflet and an outer leaflet, to exchange one or more charged donor lipids from the cyclodextrin-lipid complex to the outer leaflet under conditions suitable to form an asymmetrical large unilamellar vesicle.

In some embodiments, the present disclosure includes an asymmetrical large unilamellar vesicle made by a process including: contacting a cyclodextrin-lipid complex, including one or more charged donor lipids and cyclodextrin such as α-cyclodextrins, β-cyclodextrins, γ-cyclodextrins, or combinations thereof, or alpha, beta or gamma cyclodextrin, modified with methyl, hydroxyl or other functional groups, with a liposome including a unilamellar membrane having an inner leaflet and an outer leaflet, to exchange one or more charged donor lipids from the cyclodextrin-lipid complex to the outer leaflet under conditions suitable to form an asymmetrical large unilamellar vesicle.

### Kits

Another aspect of the present disclosure includes kits containing materials and/or instructions for the exchange of membrane lipids in liposomes to form asymmetric liposomes of the present disclosure. In embodiments, kits of the present disclosure include a cyclodextrin-lipid complex composition of the present disclosure, and optionally contain instructions for use in conjunction with the methods of the instant disclosure. The instructions may be in any suitable format, including, but not limited to, printed matter, DVD, CD, USB or directions to internet-based instructions. In embodiments, the kit may include e.g., lipids, cyclodextrin, solution, solvent, buffer, or combinations thereof.

In some embodiments, the kits include a container with or without a label. Suitable containers include, for example, bottles, vials, and test tubes. The containers may be formed from a variety of materials such as glass or plastic. In certain embodiments the kits of the present disclosure include containers, such as 15 mL conical tubes, 50 mL conical tubes, 1.5 mL centrifuge tubes, glass tubes (e.g., 10 mL), 10 cm cell culture dishes or a combination thereof. The label on the container may indicate the contents (e.g., lipids, cyclodextrin, solution, solvent, buffer) and may also indicate directions for storage, either in vivo or in vitro uses such as those described herein.

In embodiments, a kit for substituting membrane lipids in the outer leaflet of a liposome includes a container of membrane lipids such as, cationic lipids, anionic lipids, and combinations thereof, either dried or in solution, and a container that includes an amount of cyclodextrins, such as alpha-cyclodextrin or a methyl-alpha-cyclodextrin, and instructions for use. The container may be any of those known in the art and appropriate for storage and delivery of chemicals, or other biological material.

In some embodiments, kits of the present disclosure include at least one container of lipids such as, phospholipids and/or charged lipids. The lipids provided can dried (lyophilized) or in solution. In embodiments, where the lipids are in solution the lipids are dissolved in a solution including chloroform and provided in a glass container. As stated above, in certain embodiments the lipids are lipids commonly found in the liposome membrane such as, for example, lipids of the outer leaflet of the plasma membrane. For example, any lipid that includes a polar head group and acyl chain(s) can be used to form cyclodextrin-lipid complexes of the present disclosure. In specific embodiments, the lipids are exogenous phospholipids or sphingolipids. In preferred embodiments of the present disclosure, the sphingolipid is a sphingomyelin (SM) or a derivative thereof. In specific embodiments of the present disclosure, the phospholipids is phosphatidylcholine (PC) or a derivative thereof.

In some embodiments, a kit for substituting lipids in a unilamellar vesicle to form an asymmetric unilamellar vesicle is provided. In some embodiments, a kit for substituting lipids in a unilamellar vesicle to form an asymmetric unilamellar vesicle includes at least one α-cyclodextrin; at least one first instruction for forming a cyclodextrin-lipid complex including the at least one lipid bound to the α-cyclodextrin; and at least one second instruction describing a method for using the at least one cyclodextrin-lipid complex to exchange the at least one lipid between a lipid bilayer of the liposome membrane and the cyclodextrin-lipid complex to form an asymmetric unilamellar vesicle.

### Additional Embodiments

In embodiments, the cyclodextrin-lipid complex includes anionic and/or cationic lipids in accordance with the present disclosure. In embodiments, the anionic and/or cationic lipids are provided in an amount sufficient to change the charge of the outer leaflet of a liposome. In some embodiments, additional lipids and polymers that can be used to prepare liposomes for drug delivery include: dimethyldioctadecylammonium (Bromide Salt), 1,2-dimyristoyl-3-trimethylammonium-propane (chloride salt), 1,2-dipalmitoyl-3-trimethylammonium-propane (chloride salt), 1,2-stearoyl-3-trimethylammonium-propane (chloride salt), 1,2-di-O-octadecenyl-3-trimethylammonium propane (chloride salt), and combinations thereof. In embodiments, any lipid or polymers that can be used to form liposomes of the present disclosure with a plus charge, or even an ionizable functional group, such as DLin-MC3-DMA, are suitable for use herein.

In some embodiments, neutral lipids suitable for use herein include 1-palmitoyl-2-oleoyl-sn-glycero-3-phosphoethanolamine, 1,2-dioleoyl-sn-glycero-3-phosphoethanolamine lipids, and combinations thereof.

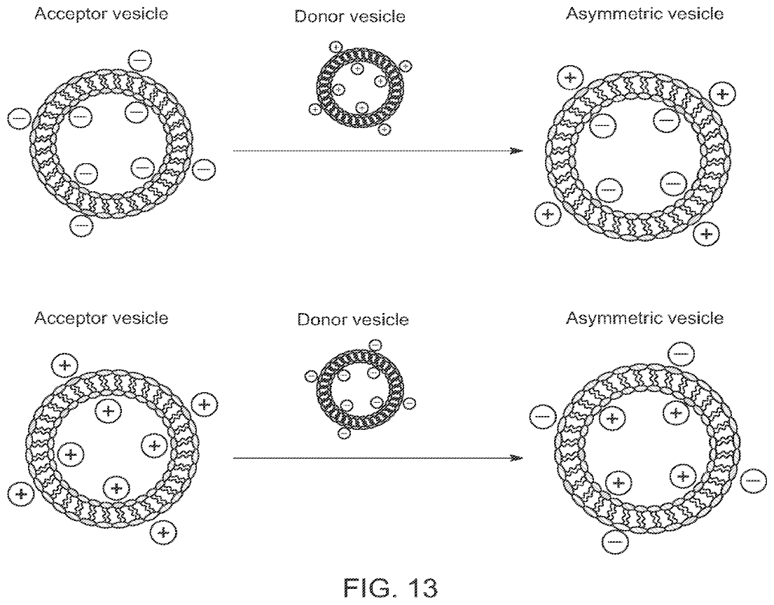

Referring now to FIG. 13, a protocol for preparing asymmetric vesicles of the present disclosure is shown. Here, a method for preparing asymmetric vesicles with positive charge on one side of the membrane and negative charge on the other is shown. In embodiments, an acceptor vesicle is shown having a net negative charge contacted with a donor vesicle having a net positive charge to form an asymmetric vesicle having a shell with a net positive outer layer and a net negative inner layer.

In embodiments, an acceptor vesicle is shown having a net positive charge contacted with a donor vesicle having a net negative charge to form an asymmetric vesicle having a shell with a net negative outer layer and a net positive inner layer.

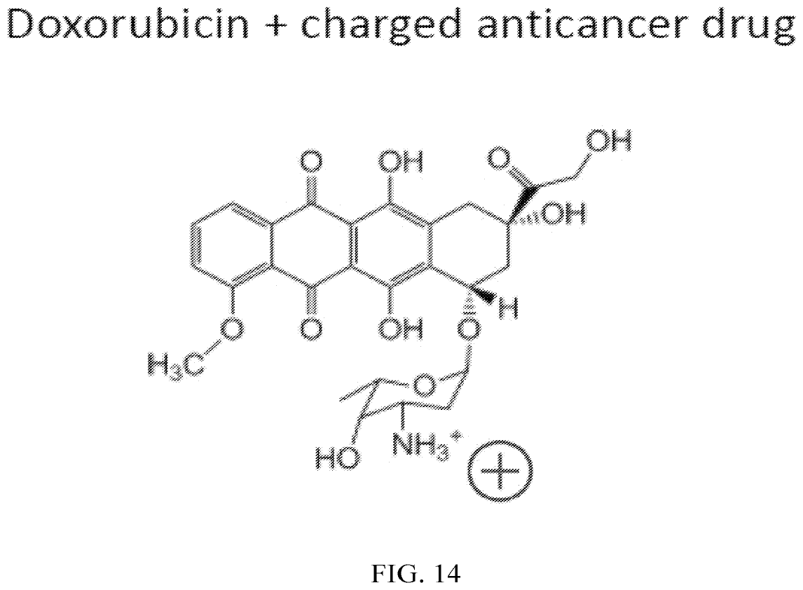

Referring now to FIG. 14, doxorubicin, a positively charged anticancer drug discussed herein is shown. It should be understood doxorubicin is a non-limiting example, and other drugs, charged drugs, including positively charged drugs are suitable for use herein.

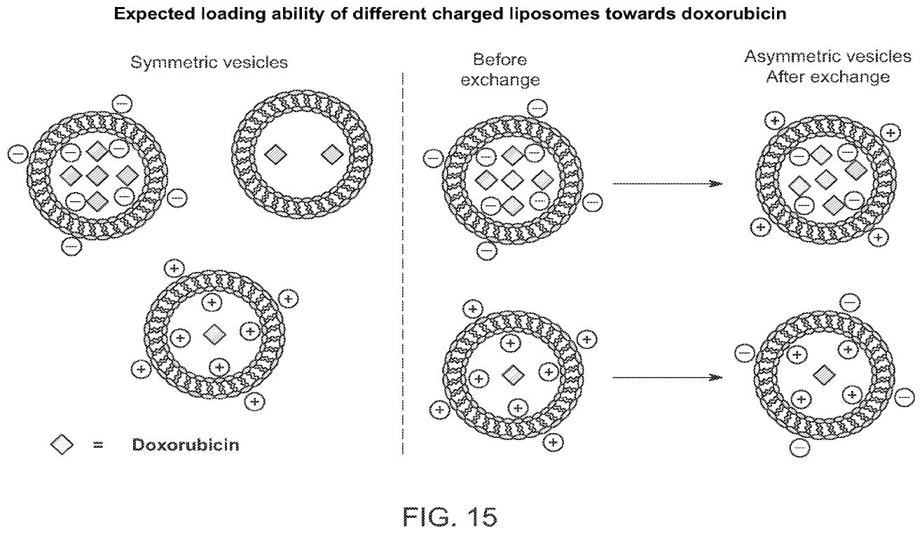

Referring to FIG. 15, a cartoon of expected loading ability of different charged liposomes towards doxorubicin is shown.

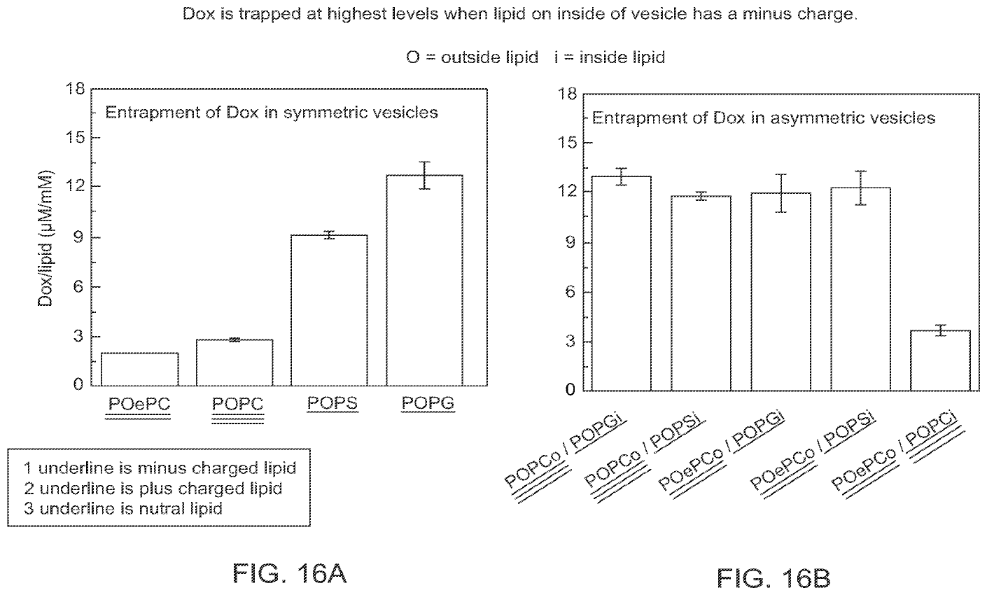

FIGS. 16A and 16B depict dox trapped at highest levels when lipid on inside of vesicle has a minus charge in accordance with the present disclosure. Various underlines are shown to depict and distinguish minus charged lipid, plus charged lipid and neutral lipid.

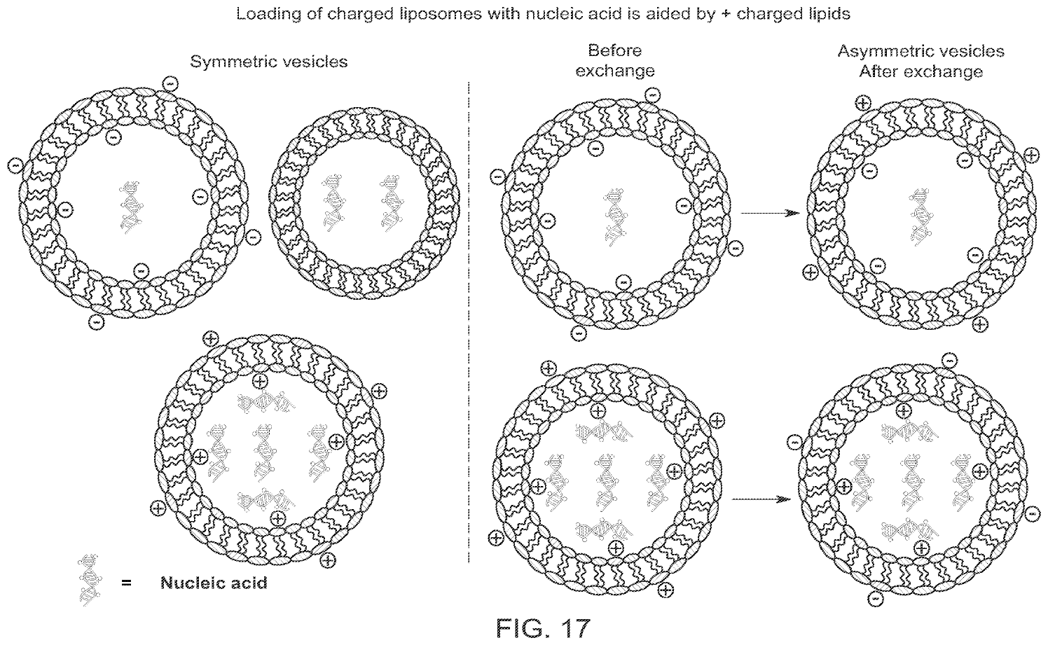

FIG. 17 depicts an embodiment of loading of charged liposomes with nucleic acid is aided by positively charged lipids. In embodiments, loading of charged liposomes with nucleic acid is aided by positively charged lipids.

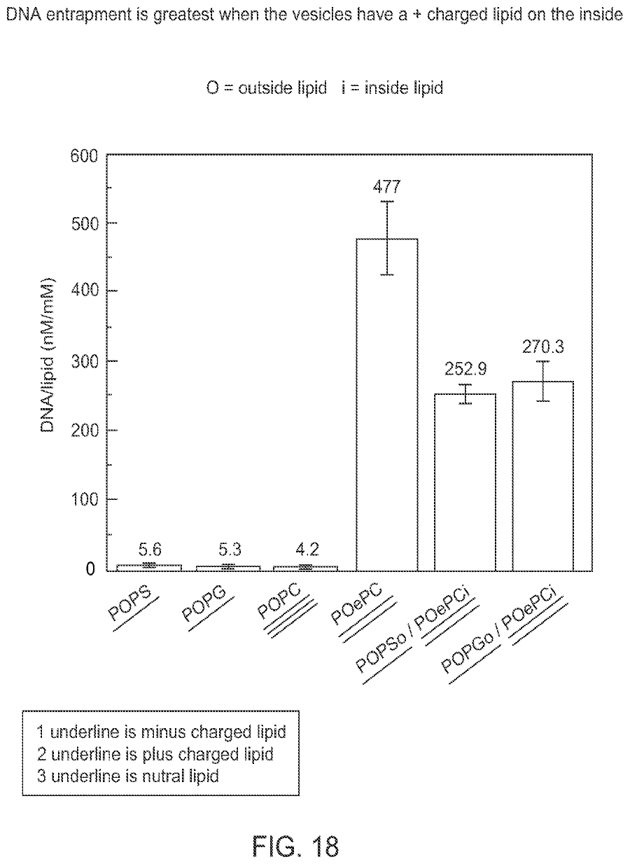

FIG. 18 depicts an embodiment, wherein DNA entrapment is greatest when vesicles have a positively charged lipid on the inside. Various underlines are shown to depict and distinguish minus charged lipid, plus charged lipid and neutral lipid.

While the invention has been shown and described with reference to certain embodiments of the present invention thereof, it will be understood by those skilled in the art that various changes in form and details may be made therein without departing from the spirit and scope of the present disclosure.

### EXAMPLES

### Example I

### Materials

1-palmitoyl-2-oleoyl-sn-glycero-3-phosphocholine (POPC), 1-palmitoyl-2-oleoyl-sn-glycero-3-ethylphosphocholine (chloride salt) (POePC), 1,2-dioleoyl-3-trimethylammonium-propane (chloride salt) (DOTAP), 1-palmitoyl-2-oleoyl-sn-glycero-3-phospho-(1′-rac-glycerol) (sodium salt) (POPG), 1-palmitoyl-2-oleoyl-sn-glycero-3-phospho-L-serine (sodium salt) (POPS), 1-palmitoyl-2-oleoyl-sn-glycero-3-phosphate (sodium salt) (POPA), and cholesterol (Choi) were purchased from Avanti Polar Lipids (Alabaster, AL). Lipids were stored in chloroform at −20° C. Concentrations were determined by dry weight. High performance thin layer chromatography (HP-TLC) plates (Silica Gel 60) were purchased from VWR International (Batavia, IL). Methyl-α-cyclodextrin (MαCD) was purchased from AraChem Cyclodextrin Shop (Tilburg, the Netherlands). It was dissolved in distilled water at close to 300 mM, and then filtered through a Sarstedt (Nümbrecht, Germany) 0.2 μm pore syringe filter. The exact concentration of MαCD was determined by comparing the refractive index of the solutions to a standard curve of refractive index vs. MαCD concentration for a known amount of MαCD dissolved in a known final volume of solution. 1(4-trimethylammoniumphenyl)-6-phenyl-1,3,5-hexatriene p-toluenesulfonate (TMADPH) was purchased from the Molecular Probes (Eugene, OR) division of Invitrogen (Carlsbad, CA). Ammonium sulfate (AS) was purchased from Fisher Scientific (Boston, MA). Doxorubicin (Dox) was purchased from Cayman Chemical (Ann Arbor, Michigan). 1,6-diphenyl-1,3,5-hexatriene (DPH) was purchased from Sigma-Aldrich (St. Louis, MO). PBS (10× phosphate-buffered saline, diluted to 1×: 10 mM sodium phosphate; and 150 mM sodium chloride, pH ˜7.4) was purchased from Bio-Rad (Hercules, CA).

### Preparation of Symmetric LUV

Lipids dissolved in chloroform were mixed in glass tubes, dried under a warm nitrogen stream and subjected to high vacuum for 1 h. The dried lipid mixtures were dispersed to 8 mM lipid concentration with 23% (w/w) sucrose in PBS (sucrose/PBS). For Dox entrapment, lipid mixtures were dispersed in sucrose/PBS with 100 μg/mL of Dox and 50 mM AS. The samples were vortexed briefly and then incubated at 37° C. for 15 min. The lipid mixtures were then cooled to room temperature and subjected to seven cycles of freeze-thaw in a liquid nitrogen bath, alternating with a 27° C. water bath. To form LUVs of uniform vesicle size, the lipid mixtures were then extruded 11 times through 100 nm-pore polycarbonate membranes (Sigma-Aldrich, St. Louis, MO).

If needed to wash away external sucrose (e.g. to prepare acceptor vesicles for lipid exchange or prepare samples for size measurements), 200 μL aliquots of LUV were mixed with 3.8 mL PBS and pelleted by ultracentrifugation at 190,000×g for 30 min at 23° C. using a Beckman L8-80M ultracentrifuge with a SW-60 rotor. Following pelleting, the supernatant was removed, the LUV containing-pellet dispersed in 0.5 mL PBS. When samples had entrapped Dox they were re-centrifuged twice with 4 mL PBS using the same protocol. Finally, the LUV pellet was dispersed in 500 μL PBS, covered with aluminum foil, and reserved for use. Unless otherwise noted samples were used within 2 h of preparation.

### Preparation of Donor Lipid-Loaded MαCD for Lipid Exchange Experiments

Desired ratios of charged lipids (POePC, DOTAP, POPS, POPG or POPA) and zwitterionic POPC dissolved in chloroform were combined in glass tubes, dried under a warm nitrogen stream, and then subjected to high vacuum for 1 h. The dried lipids were placed in a 70° C. water bath and dispersed at 70° C. with an aliquot of pre-warmed PBS, and then an aliquot of pre-warmed MαCD, to give a final concentration of 40 mM MαCD and 16 mM lipid. The samples were vortexed briefly, and then vortexed in a multitube vortexer for 2 h at 55° C., cooled to room temperature, covered in foil, and reserved for further use.

### Preparation of Acceptor LUV for Lipid Exchange Experiments

The desired amount of charged lipids (POePC, DOTAP, POPS, POPG or POPA) (between 15-30 mol % of total lipid), zwitterionic POPC (30-45 mol % of total lipid), and cholesterol (40 mol % of total lipid) dissolved in chloroform were combined in glass tubes. LUVs were then prepared as described above for symmetric vesicles.

### Outer Leaflet Lipid Exchange

To wash away untrapped sucrose from acceptor LUVs, 500 μL aliquots of acceptor lipid were diluted with 3.5 ml 1×PBS and subjected to ultracentrifugation at 190,000 g for 30 min at 23° C. as above. The supernatant was discarded, the LUV pellets were resuspended to 8 mM lipid concentration with 1×PBS and used immediately. To exchange the outer leaflet of acceptor LUV, 500 μL of the donor lipid-MαCD mixture and 500 μL of the acceptor lipid mixtures were combined, covered in foil, and shaken for 45 min at 37° C. These lipid-exchange mixtures were layered over 3 mL 7.4% (w/w) sucrose dissolved in 4×PBS and subjected to ultracentrifugation at 190,000×g for 45 min at 23° C. Following centrifugation, most of the supernatant was carefully removed, leaving approximately 750 μL sucrose/4×PBS and loosely pelleted asymmetric LUVs in the bottom of the centrifuge tube. The upper portion of the tube was swabbed with a clean, dry cotton tipped applicator to remove residual adhering donor lipids and MαCD. Approximately 3.25 mL PBS was then added to the tube and thoroughly mixed with asymmetric LUVs and residual supernatant. This mixture was centrifuged a second time as above for 30 min. Following centrifugation, all remaining supernatant was removed, and the pellet was dispersed for immediate use in up to 500 μL PBS or distilled water if samples were for TLC analysis. The asymmetric LUVs lipid concentration was determined by HP-TLC or DPH assay (see below in Methods) and the mean yield was ˜10.5% of theoretical maximal yield, see Table 1 below), with a final lipid concentration 0.83±0.22 mM. Entrapped Dox did not appear to reproducibly affect asymmetric LUVs lipid yield.

TABLE 1
(Asymmetric LUVs lipid yield. Yield is percent of lipid relative
to the initial amount of acceptor lipid used in the preparation).

| Asymmetric LUVs | Lipid Yield |
|---|---|
| POePC:POPC out/POPC in/Chol | 8.6% ± 1.3% |
| POPG:POPC out/POPC in/Chol | 8.3% ± 1.0% |
| POPS:POPC out/POPC in/Chol | 13.9% ± 2.1% |
| POePC:POPC out/POPG:POPC in/Chol | 12.0% ± 1.3% |
| POePC:POPC out/POPS:POPC in/Chol | 13.0% ± 2.1% |
| POPS:POPC out/POePC:POPC in/Chol | 13.0% ± 2.1% |
| POPG:POPC out/POePC:POPC in/Chol | 9.9% ± 1.2% |

### High-Performance TLC (HP-TLC)

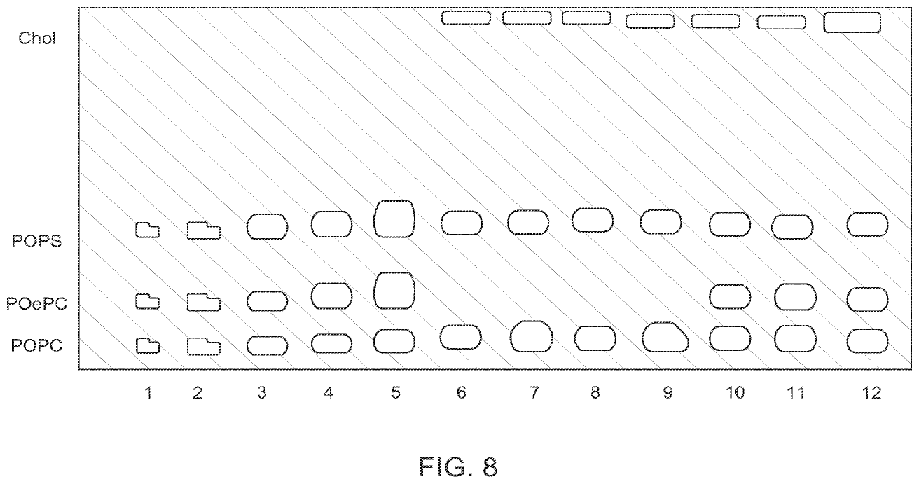

Aliquots of samples and lipid standards were dissolved in 1:1 (v/v) chloroform/methanol. Dissolved lipids were applied to HP-TLC (Silica Gel 60) plates (Merck) and chromatographed to within 20% of full plate height in 3:1:1 chloroform:methanol:acetic acid (v/v). After chromatography, the plates were air dried, saturated with 3% (w/v) cupric-acetate-8% (v/v) phosphoric acid by spraying, and then air-dried again. Plates were then charred on a hot place at ˜180° C. to develop lipid bands. Lipid band intensity was measured using ImageJ software (National Institutes of Health). Lipids in samples were quantified by comparing background-subtracted band intensity with that of various standard amounts of each lipid chromatographed on the same TLC plate. A sample TLC plate is shown in FIG. 8. The intensity in the standard bands was fit to a linear intensity vs. lipid quantity curve using Excel software (Microsoft Corporation, Redmond, WA).

### Fluorescence Measurements

Fluorescence measurements were carried out using a SPEX FluoroLog 3 spectrofluorometer (Horiba Scientific, Edison, New Jersey) using quartz semimicro cuvettes (excitation pathlength, 10 mm; emission pathlength, 4 mm). DPH fluorescence was measured at an excitation wavelength of 358 nm and emission wavelength of 430 nm. TMADPH fluorescence was measured at an excitation wavelength of 364 nm and emission wavelength of 426 nm. Dox fluorescence was measured at an excitation wavelength of 470 nm and emission wavelength of 595 nm. The slit bandwidths were set to 3 mm (about 5 nm bandpass) for both excitation and emission. Fluorescence was measured at room temperature. Background samples, which lacked fluorescent probe, had negligible intensity (<1% of samples with fluorescent probe), except in DPH fluorescence anisotropy measurements.

For fluorescence anisotropy, background fluorescence values (with polarization filters in various positions) were measured first in LUV samples without DPH, then DPH was added and fluorescence measured as described previously (See e.g., Natchiar, S. K., Myasnikov, A. G., Kratzat, H., Hazemann, I. & Klaholz, B. P. Visualization of chemical modifications in the human 80S ribosome structure. Nature 551, 472-477 (2017)). Background values for each combination of polarization filter positions were subtracted from the values after DPH was added, and anisotropy calculated.

### Fluorescence Measurement of Doxorubicin Concentration

It was found that Dox trapped in LUVs did not have the same fluorescence intensity as when dissolved in solution, likely because it is present within liposomes in an aggregate having a decreased quantum yield. When vesicles with entrapped Dox were dissolved with 1 (v/v) % Triton X-100 Dox fluorescence increased to a level about the same as Dox dissolved in PBS. (Addition of Triton X-100 did not affect the fluorescence of Dox in PBS.) Dissolving the vesicles with 1% Triton increased Dox fluorescence by about 150%, 15%, and 45% when the Triton X-100 was added to cationic, neutral, and anionic vesicles containing trapped Dox, respectively. This was used as a correction factor to convert the intensity of fluorescence for vesicle entrapped Dox to that which would be measured for Dox in solution.

### Fluorescence Measurement of Lipid Concentration and Outer Leaflet Charge

Lipid concentration in asymmetric LUVs and symmetric LUVs after centrifugation was estimated via TLC or the level of DPH bound as measured by fluorescence. To do the latter a standard linear curve of fluorescence vs. lipid concentration was prepared using symmetric LUVs with POPC, 40 mol % cholesterol and with or without 15 mol % of the charged lipids used in the LUVs to be assayed. (However, it should be noted that the standard curves were not affected by the presence or absence of 15 mol % charged phospholipid, and so were averaged to give the final standard curve.) Standard samples were diluted with PBS to the desired concentration.

Experimental samples for which lipid concentration was to be determined were diluted 100 to 400-fold (to ˜2-8 μM lipid) by adding aliquots of each sample to quartz semi-micro cuvettes containing enough of PBS to give a total volume of 1 ml and then 20 μL of 18 μM DPH dissolved in ethanol added. To test the lipid concentration of symmetric or asymmetric LUVs with Dox entrapped inside, standard samples were prepared the same way except the lipids were hydrated with 100 μg/mL of Dox and 50 mM AS.

For the TMA-DPH binding assay, samples were diluted to 118 or 257 μM by adding aliquots of LUVs to quartz semi-micro cuvettes containing enough of either PBS or sucrose/PBS to give a total volume of 1 ml, and then 20 μL of 10 μM TMA-DPH dissolved in ethanol was added. The final TMA-DPH concentration was 0.2 μM.

A standard curve was prepared from symmetric LUVs composed of POPC, various amounts up to 15 mol % of the charged lipids of interest and 40 mol % cholesterol. Lipid concentrations for the standard curve were 118 or 257 μM. From the standard curve samples a graph of TMA-DPH fluorescence (F) normalized to that of 0.2 μM TMA-DPH dissolved in ethanol (F₀) vs. net % of charged phospholipid. A fourth order polynomial fit was used for the standard curve. Experimental asymmetric LUVs samples were diluted with PBS to match lipid concentration to that in the standard curve. The value of fluorescence was then normalized to the fluorescence of 0.2 μM TMADPH in ethanol, and the value obtained was compared to the standard curve.

### Measurement of Dynamic Light Scattering

LUV size was determined by dynamic light scattering using a ProteinSolution DynaPro instrument (Wyatt Technology, Santa Barbara, CA) at 20° C. AUVs and symmetric LUVs were diluted to —50-80 μM using PBS filtered with a 0.2-15 mm-filter. Vesicle sizes were estimated with the use of the Dynamics V5.25.44 program supplied by Wyatt Technology. Acceptor vesicles before and after exchange of outer-leaflet lipids had similar diameters of 125±20 nm.

### Results

### Preparation and Final Phospholipid Composition of Asymmetric Vesicles

A MαCD-mediated lipid exchange method (See e.g., Li, G.; Kim, J.; Huang, Z.; Clair, J. R. S.; Brown, D. A.; London, E., Efficient replacement of plasma membrane outer leaflet phospholipids and sphingolipids in cells with exogenous lipids. Proceedings of the National Academy of Sciences 2016, 113 (49), 14025-14030; and Clair, J. W. S.; London, E., Effect of sterol structure on ordered membrane domain (raft) stability in symmetric and asymmetric vesicles. Biochimica et Biophysica Acta (BBA)-Biomembranes 2019, 1861 (6), 1112-1122) was adapted here to application to cationic lipids, and to prepare asymmetric LUVs with opposite net charge on their inner and outer leaflets. In the exchange protocol MαCD exchanges phospholipids from donor vesicles with one lipid composition with the phospholipids in the outer leaflet of acceptor LUVs having a different lipid composition, converting the acceptor LUVs into asymmetric LUVs. The acceptor vesicles contain trapped sucrose to aid isolation by centrifugation, and the desired concentration of cholesterol, which is not exchanged by MαCD and so remains in the asymmetric LUVs. When the donor lipid is in excess, the final asymmetric LUVs formed from the acceptor LUVs have an outer leaflet with a phospholipid composition similar to that of the donor vesicles prior to exchange (See e.g., Cheng, H.-T.; London, E., Preparation and properties of asymmetric vesicles that mimic cell membranes effect upon lipid raft formation and transmembrane helix orientation. Journal of Biological Chemistry 2009, 284 (10), 6079-6092; Cheng, H.-T.; London, E., Preparation and properties of asymmetric large unilamellar vesicles: interleaflet coupling in asymmetric vesicles is dependent on temperature but not curvature. Biophysical journal 2011, 100 (11), 2671-2678) and (FIG. 1).

Preliminary studies showed yields were highest and most consistent when acceptor vesicles contained 40 mol % cholesterol (relative to vesicles with less or no cholesterol) and when charged lipids were no more than 25 mol % of the total phospholipid (equal to 15 mol % of total lipid). The remainder of the phospholipid used was POPC (45 mol % of total lipid, which is equal to 75 mol % of phospholipid). One change from previous protocols was that to obtain a high yield of vesicles after centrifugation, a higher concentration of PBS was used in the sucrose solution in which centrifugation was carried out. Referring to FIG. 1, a schematic illustration of asymmetric LUV preparation is shown.

Using these conditions, asymmetric LUVs with a range of charged lipid compositions were prepared. Symmetric LUVs prepared similarly to the asymmetric vesicles but without a lipid exchange step were also prepared. The symmetric vesicles were composed of POPC, 40 mol % cholesterol and, when desired, 15 mol % of cationic lipid (POePC or DOTAP) or of anionic phospholipid (POPG. POPS or POPA). (Note: although DOTAP is not a phospholipid, for simplicity, when talking about lipids other than cholesterol we will use the term “phospholipid” below instead of the more precise “phospholipid or DOTAP”.) Asymmetric LUVs were prepared in which the outer leaflet was cationic or anionic, and the inner leaflet had the opposite charge or was uncharged.

Without wishing to be bound by any theory, if exchange was 100% complete, in the asymmetric LUVs the inner leaflet would have the phospholipid composition of the acceptor vesicle and the outer leaflet would have the phospholipid composition of the donor vesicle. Cholesterol would be in both leaflets because, as noted above, it is not exchanged out of the acceptor vesicles by MαCD. The actual extent of exchange can be influenced by phospholipid structure, which can modulate binding to MαCD, and any differential ability of phospholipids to be extracted from or inserted into lipid vesicles. This is likely to be dependent upon both the structure of the lipids being exchanged and of the lipid composition of the vesicles from which the lipid is being removed or inserted.

Table 2 shows the composition of the donor and acceptor vesicles used for lipid exchange. For example, Table 2 provides phospholipid composition of asymmetric LUVs. Cholesterol content of acceptor vesicles and asymmetric vesicles is ˜40mol %. *Note that due to inefficient exchange using POPS:POPC as the donor and acceptor containing DOTAP and POPC did NOT result in production of asymmetric LUVs with net anionic outer leaflets.

TABLE 2

| | | | |
|---|---|---|---|
| | | | Calculated |
| | | | Outer Leaflet |
| | | Phospholipid | Phospholipid |
| Donor | Acceptor | Composition | Composition |
| Phospholipid | Phospholipid | in Asymmetric | in Asymmetric |
| Composition | Composition | Vesicles | Vesicles |

Charged Outside/Neutral Inside

| | | | |
|---|---|---|---|
| POePC:POPC | POPC | POePC:POPC | POePC:POPC |
| 25:75 | | 8:92 | 17:83 |
| DOTAP:POPC | POPC | DOTAP:POPC | DOTAP:POPC |
| 25:75 | | 9:91 | 17:83 |
| POPS:POPC | POPC | POPS:POPC | POPS:POPC |
| 25:75 | | 12:88 | 23:77 |
| POPG:POPC | POPC | POPG:POPC | POPG:POPC |
| 25:75 | | 9:91 | 18:82 |
| POPA:POPC | POPC | POPA:POPC | POPA:POPC |
| 25:75 | | 12:88 | 24:76 |

Anionic Outside/Cationic Inside

| | | | |
|---|---|---|---|
| POPS:POPC | POePC:POPC | POPS:POePC:POPC | POPS:POePC:POPC |
| 25:75 | 25:75 | 11:16:73 | 22:7:71 |
| POPG:POPC | POePC:POPC | POPG:POePC:POPC | POPG:POePC:POPC |
| 25:75 | 25:75 | 12:18:70 | 25:10:65 |
| POPA:POPC | POePC:POPC | POPA:POePC:POPC | POPA:POePC:POPC |
| 25:75 | 25:75 | 8:13:79 | 16:1:83 |
| POPS:POPC | DOTAP:POPC | POPS:DOTAP:POPC | POPS:DOTAP:POPC* |
| 25:75 | 25:75 | 6:22:72 | 12:19:69 |
| POPG:POPC | DOTAP:POPC | POPG:DOTAP:POPC | POPG:DOTAP:POPC |
| 25:75 | 25:75 | 11:14:75 | 21:4:75 |

Cationic Outside/Anionic Inside

| | | | |
|---|---|---|---|
| POePC:POPC | POPS:POPC | POePC:POPS:POPC | POePC:POPS:POPC |
| 25:75 | 25:75 | 11:14:75 | 23:2:75 |
| DOTAP:POPC | POPS:POPC | DOTAP:POPS:POPC | DOTAP:POPS:POPC |
| 25:75 | 25:75 | 10:17:73 | 20:10:70 |
| POePC:POPC | POPG:POPC | POePC:POPG:POPC | POePC:POPG:POPC |
| 25:75 | 25:75 | 12:21:67 | 24:17:59 |
| DOTAP:POPC | POPG:POPC | DOTAP:POPG:POPC | DOTAP:POPG:POPC |
| 25:75 | 25:75 | 7:19:74 | 14:12:74 |
| POePC:POPC | POPA:POPC | POePC:POPA:POPC | POePC:POPA:POPC |
| 25:75 | 25:75 | 8:13:79 | 16:1:83 |

To experimentally determine the actual efficiency of exchange, the phospholipid composition of the vesicles was assayed by quantitative TLC. Average (mean) values for phospholipid composition in vesicles after exchange are summarized in Table 2 (results with both the mean values and standard deviations are shown in Tables 3 and 4).

Referring to Table 3, a lipid composition of asymmetric LUVs was determined by TLC. Values shown are mean and standard deviation from three preparations.

TABLE 3

| | Acceptor | | Cationic | Anionic |
|---|---|---|---|---|
| | Lipid | Neutral | Lipid | Lipid |
| | Composition | Lipid | mol % | mol % |
| Donor Lipid | (w/40 mol % | mol % | (POePC or | (POPG or |
| Composition | Chol) | (POPC) | DOTAP) | POPS) |
| POePC:POPC | POPC | 91.7 ± 2.7 | 8.3 ± 2.7 | |
| 25:75 | | | | |
| POPS:POPC | POPC | 91.4 ± 1.3 | 8.6 ± 1.3 | |
| 25:75 | | | | |
| POPG:POPC | POPC | 88.5 ± 2.4 | | 11.5 ± 2.4 |
| 25:75 | | | | |
| DOTAP:POPC | POPC | 90.8 ± 1.7 | | 9.2 ± 1.7 |
| 25:75 | | | | |
| POPA:POPC | POPC | 87.9 ± 3.5 | | 12.1 ± 3.5 |
| 25:75 | | | | |
| POPS:POPC | POePC:POPC | 73.0 ± 2.3 | 15.8 ± 0.8 | 11.2 ± 2.2 |
| 25:75 | 25:75 | | | |
| POPG:POPC | POePC:POPC | 70.2 ± 2.9 | 17.5 ± 2.5 | 12.3 ± 1.8 |
| 25:75 | 25:75 | | | |
| POPA:POPC | POePC:POPC | 79.0 ± 2.6 | 12.8 ± 1.3 | 8.2 ± 3.1 |
| 25:75 | 25:75 | | | |
| POPS:POPC | DOTAP:POPC | 72.0 ± 1.8 | 22.1 ± 0.5 | 5.9 ± 1.8 |
| 25:75 | 25:75 | | | |
| POPG:POPC | DOTAP:POPC | 75.0 ± 2.3 | 14.5 ± 1.3 | 10.5 ± 2.5 |
| 25:75 | 25:75 | | | |
| POePC:POPC | POPS:POPC | 75.0 ± 1.2 | 11.3 ± 1.5 | 13.7 ± 2.3 |
| 25:75 | 25:75 | | | |
| DOTAP:POPC | POPS:POPC | 72.5 ± 4.8 | 10.0 ± 1.1 | 17.5 ± 4.7 |
| 25:75 | 25:75 | | | |
| POePC:POPC | POPG:POPC | 67.3 ± 4.6 | 11.8 ± 1.0 | 21.0 ± 4.8 |
| 25:75 | 25:75 | | | |
| DOTAP:POPC | POPG:POPC | 74.3 ± 1.9 | 7.2 ± 0.8 | 18.5 ± 1.7 |
| 25:75 | 25:75 | | | |
| POePC:POPC | POPA:POPC | 79.1 ± 1.9 | 8.1 ± 1.1 | 12.8 ± 1.8 |
| 25:75 | 25:75 | | | |

Referring now to Table 4, lipid composition of outer leaflet of asymmetric LUVs calculated from TLC results is shown. Values shown are mean and standard deviation from three preparations.

TABLE 4

| | Acceptor | | Cationic | Anionic |
|---|---|---|---|---|
| | Lipid | Neutral | Lipid | Lipid |
| | Composition | Lipid | mol % | mol % |
| Donor Lipid | (w/40 mol % | mol % | (POePC or | (POPG, POPS |
| Composition | Chol) | (POPC) | DOTAP) | or POPA) |
| POePC:POPC | POPC | 83.3 ± 5.3 | 16.7 ± 5.3 | |
| 25:75 | | | | |
| POPS:POPC | POPC | 82.8 ± 2.7 | 17.2 ± 2.7 | |
| 25:75 | | | | |
| POPG:POPC | POPC | 77.0 ± 4.9 | | 23.0 ± 4.9 |
| 25:75 | | | | |
| DOTAP:POPC | POPC | 81.6 ± 3.4 | | 18.4 ± 3.4 |
| 25:75 | | | | |
| POPA:POPC | POPC | 75.8 ± 7.0 | | 24.3 ± 7.0 |
| 25:75 | | | | |
| POPS:POPC | POePC:POPC | 70.3 ± 4.7 | 6.7 ± 1.5 | 22.3 ± 4.5 |
| 25:75 | 25:75 | | | |
| POPG:POPC | POePC:POPC | 65.5 ± 3.5 | 10.3 ± 1.0 | 24.5 ± 3.5 |
| 25:75 | 25:75 | | | |
| POPA:POPC | POePC:POPC | 84.5 ± 5.2 | 1.0 ± 2.6 | 16.8 ± 6.2 |
| 25:75 | 25:75 | | | |
| POPS:POPC | DOTAP:POPC | 69.0 ± 5.9 | 19.5 ± 5.1 | 11.8 ± 3.6 |
| 25:75 | 25:75 | | | |
| POPG:POPC | DOTAP:POPC | 75.4 ± 4.7 | 4.0 ± 2.5 | 21.0 ± 5.1 |
| 25:75 | 25:75 | | | |
| POePC:POPC | POPS:POPC | 74.7 ± 2.3 | 22.3 ± 2.9 | 2.3 ± 4.7 |
| 25:75 | 25:75 | | | |
| DOTAP:POPC | POPS:POPC | 70.0 ± 9.5 | 20.0 ± 2.1 | 10.0 ± 9.4 |
| 25:75 | 25:75 | | | |
| POePC:POPC | POPG:POPC | 59.5 ± 9.1 | 23.5 ± 1.9 | 17.0 ± 9.5 |
| 25:75 | 25:75 | | | |
| DOTAP:POPC | POPG:POPC | 73.6 ± 3.8 | 14.4 ± 1.6 | 12.4 ± 3.4 |
| 25:75 | 25:75 | | | |
| POePC:POPC | POPA:POPC | 83.3 ± 3.8 | 16.3 ± 2.3 | 0.5 ± 3.7 |
| 25:75 | 25:75 | | | |

The calculated outer leaflet compositions are shown in rightmost column of Table 2. The composition of the outer leaflet was calculated based on prior observations showing that: 1) exchange is specific to the outer leaflet; and 2) phospholipid flip-flop between leaflets, which would destroy asymmetry, is very slow (days) for the types of lipid compositions studied here (See e.g., Cheng, H.-T.; London, E., Preparation and properties of asymmetric vesicles that mimic cell membranes effect upon lipid raft formation and transmembrane helix orientation. Journal of Biological Chemistry 2009, 284 (10), 6079-6092; Lin, Q.; London, E., Preparation of artificial plasma membrane mimicking vesicles with lipid asymmetry. PloS one 2014, 9 (1); Son, M.; London, E., The dependence of lipid asymmetry upon polar headgroup structure. Journal of lipid research 2013, 54 (12), 3385-3393; Dicorleto, P. E.; Zilversmit, D. B., Exchangeability and rate of flip-flop of phosphatidylcholine in large unilamellar vesicles, cholate dialysis vesicles, and cytochrome oxidase vesicles. Biochimica et Biophysica Acta (BBA)-Biomembranes 1979, 552 (1), 114-119; Johnson, L.; Hughes, M.; Zilversmit, D., Use of phospholipid exchange protein to measure inside-outside transposition in phosphatidylcholine liposomes. Biochimica et Biophysica Acta (BBA)-Biomembranes 1975, 375 (2), 176-185; and Rothman, J. E.; Dawidowicz, E. A., Asymmetric exchange of vesicle phospholipids catalyzed by the phosphatidylcholine exchange protein. Measurement of inside-outside transitions. Biochemistry 1975, 14 (13), 2809-2816). The expected value for 100% efficient replacement of acceptor phospholipid with donor phospholipid would be an outer leaflet in which 25 mol % of total outer leaflet phospholipid would be charged lipid from the donor, assuming charged phospholipid and POPC exchange with equal efficiency.

A significant amount of exchange was achieved when donor lipid contained various combinations of different cationic and anionic lipids with POPC and acceptor vesicles contained POPC and cholesterol. The % of charged donor phospholipid in the asymmetric vesicles prepared from the acceptor vesicles was ˜60-95 mol % of the value for complete exchange. There was slightly lower mean exchange efficiency for donor containing cationic phospholipid (˜70%) than for donor containing anionic phospholipid (˜88%).

The pattern for the efficiency of charged phospholipid exchange asymmetric LUVs when the both the donor and acceptor contained charged phospholipid was somewhat different. There was a relatively high value of efficiency of charged donor phospholipid exchanged into the acceptor vesicles in most cases (˜80-100%), but also a few cases with relatively low efficiency of exchange (˜50-65%). This had consequences for which types of asymmetric LUVs can be prepared with strongly opposite net charge in each leaflet (see below).

The efficiency of exchange of POPC relative to charged phospholipid is an additional parameter that influences final phospholipid composition. If exchange of POPC and charged phospholipids between donor and acceptor were equally efficient in the samples in which both donor and acceptor contain 25 mol % charged phospholipid, the final fraction of POPC in the phospholipid of the asymmetric LUVs would be the same as before exchange, 75 mol %. The mean experimental values for POPC content as a percent of total phospholipid in experiments in which both donor and acceptor had charged phospholipids was 74 mol %. This indicates that the relative exchange efficiencies of charged lipid and POPC are similar, although some phospholipid compositions resulted in slightly lower (˜60 mol %) or higher (˜85 mol %) POPC content in total phospholipid, suggestive of slightly different exchange efficiencies for POPC and phospholipids with net charge.

Somewhat unequal (non-random) exchange of different phospholipids is also suggested by the fact that in some experiments in which donor and acceptor both contained 25 mol % charged phospholipid, the final total mol % of phospholipid that was charged in the asymmetric vesicles was somewhat less than or greater than 25 mol %. The former case occurs when charged phospholipid from the donor is not exchanged as efficiently as donor POPC, and/or charged phospholipid from the acceptor is exchanged more efficiently than acceptor POPC. The latter case occurs when charged phospholipid is extracted from acceptor vesicles less efficiently than acceptor POPC, and/or when charged phospholipid is exchanged into acceptor more efficiently than POPC. In such cases, the outer leaflet contains substantial amounts of both anionic and cationic lipid. Indeed, in some cases the outer leaflet retained a significant amount of charged lipid from the acceptor vesicles showing that there was inefficient exchange of charged acceptor phospholipid relative to exchange of acceptor POPC, and/or lipid exchange was not complete.

Despite these complications, the overall level of exchange in most cases was sufficient to prepare a wide variety of asymmetric LUVs with different signs on the net charges on their inner and outer leaflet (Table 2). One exception was the case in which the donor contained POPS and acceptor DOTAP, in which the level of exchange was so low, that the outer leaflet of the acceptor vesicles remained cationic before and after exchange. Another example of poor exchange was when the donor contained DOTAP and the acceptor POPG. In that case the level of exchange was only enough to result in a near-neutral outer leaflet rather than the desired highly cationic outer leaflet.

It should be noted that DPH anisotropy, which measures membrane order, was not significantly different for the different preparations of symmetric and asymmetric vesicles (data not shown). This indicates that for the lipids used, charge and asymmetry did not affect membrane order.

### TMADPH Assay to Measure Charge in the Outer Leaflet of Asymmetric LUV

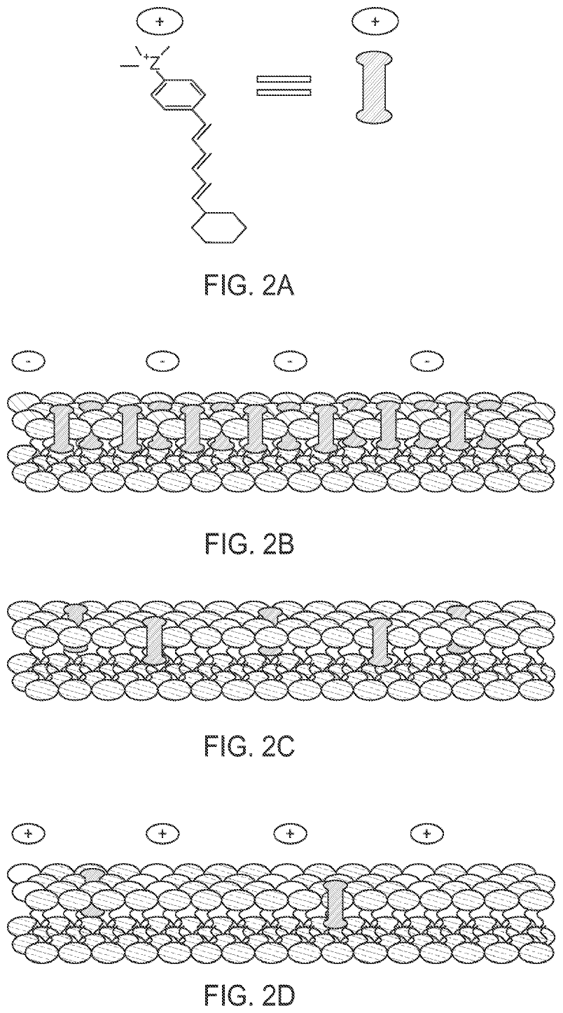

Although the outer leaflet phospholipid compositions estimated from the extent of lipid exchange should be valid given the prior demonstration that phospholipid exchange only involves the outer leaflet and lipid flip-flop between leaflets is slow, as noted above, it was desirable to have a confirmatory method to estimate the charge on the outer leaflet of the asymmetric LUVs. To achieve this a novel TMA-DPH binding assay was developed. The structure of the cationic fluorescent probe TMA-DPH is shown in the FIG. 2A. Because TMA-DPH is cationic and does not rapidly cross membranes ^{19, 32-33}, its binding to and insertion into membranes is dependent on outer leaflet charge, with a higher level of binding to anionic membranes, as shown schematically in FIG. 2B. After inserting into the hydrophobic core of the vesicle bilayer, the fluorescence of TMA-DPH greatly increases, which allows facile detection of binding.

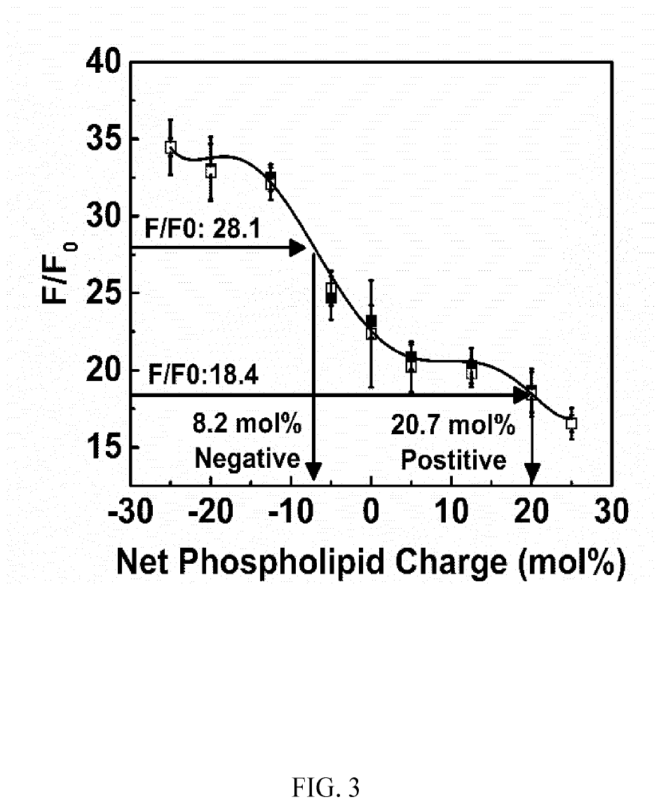

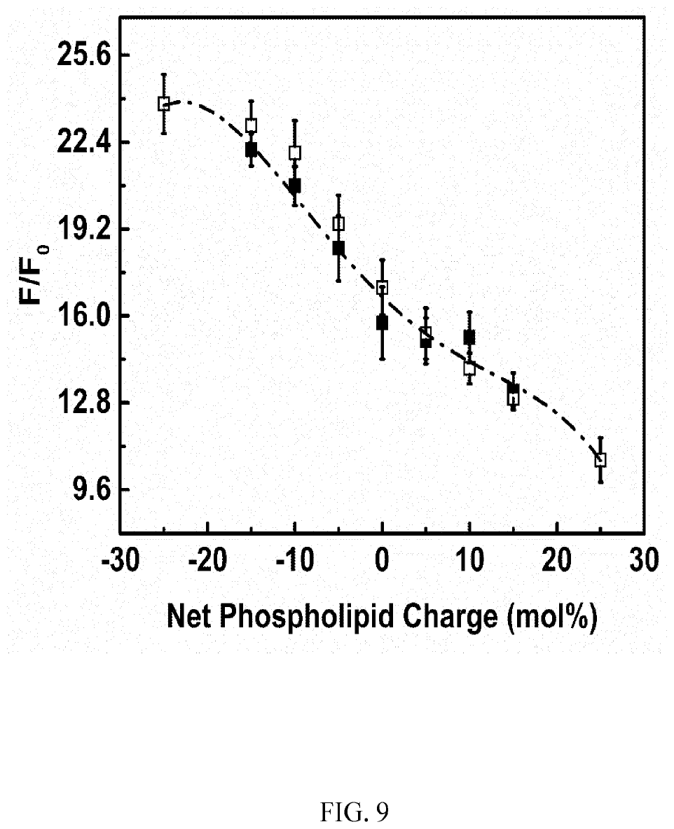

FIG. 3 shows an example of a standard curve for estimating charged lipid content in the outer leaflet of LUVs. The standard curve shown is for LUVs composed of 40 mol % cholesterol and mixtures of POPC, POPS, and POePC. Other lipid mixtures gave similar standard curves (FIG. 9). Two types of standard curves were prepared. In one set the standard curve samples were composed of (in addition to cholesterol) binary phospholipid mixtures containing various ratios of POPC and POPS, to prepare standard samples with 0-25% net negative charged phospholipid, or various ratios of POPC and POePC, to prepare standard samples with 0-25 mol % net positively charged phospholipid. In the second set of standards cholesterol and a ternary phospholipid mixture of POPC, POPS, and POePC was used, in which total charged phospholipid was fixed at 25 mol % of total phospholipid, but with different ratios of POPS to POePC. To illustrate the difference between these two sets of samples, in the first set, the samples with zero net charged phospholipid contained only cholesterol and POPC, while in the second set the samples with zero net charged phospholipid contained cholesterol and phospholipid composed of 75 mol % POPC, 12.5 mol % POPS and 12.5 mol % POePC.

As shown FIG. 3, the two sets of standard samples gave almost identical similar TMA-DPH fluorescence values for vesicles having equivalent net charge, indicating that TMA-DPH binding was simply sensitive to net phospholipid charge. Similar behavior was observed for the standard curves prepared for other mixtures of cholesterol with binary and ternary phospholipid mixtures (FIG. 9). FIG. 3 also shows experimental values for TMA-DPH fluorescence for POPS:POPC out/POePC:POPC in/Chol and for POePC:POPC out/POPS:POPC in/Chol asymmetric LUV, and illustrates how these values were used to estimate outer leaflet charge. The values for net outer leaflet charge for these vesicles was 8.2 mol % negative charge and 20.7 mol % positive charge, respectively.

### Comparison of TLC and TMA-DPH Assay of Outer Leaflet Charge

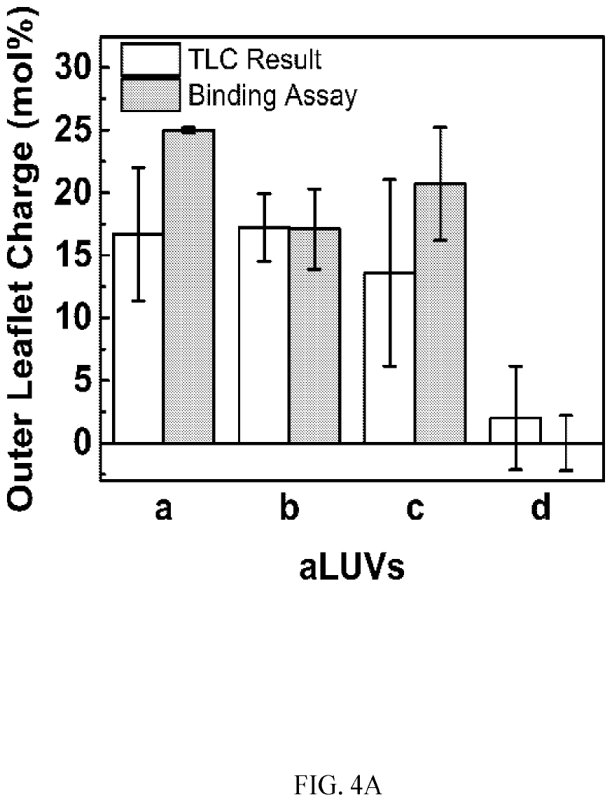

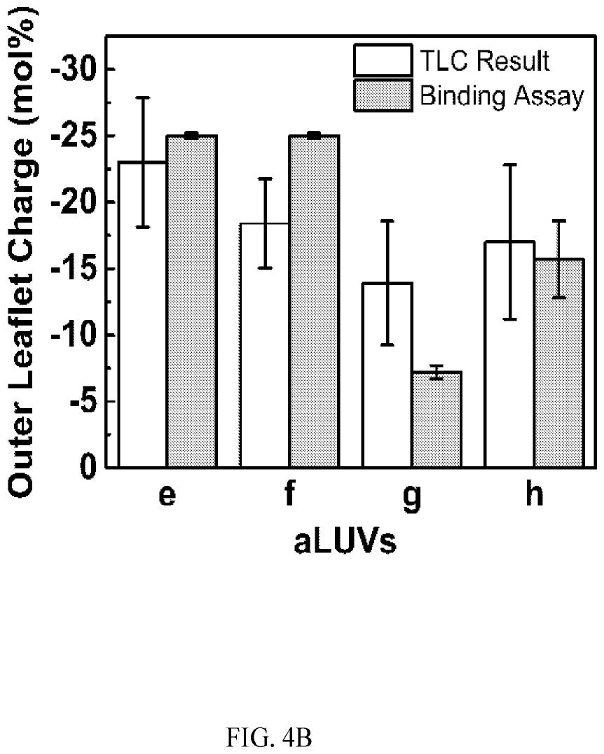

FIG. 4 compares the results for outer leaflet charge from the TMA-DPH assay to the values estimated from the phospholipid composition of the asymmetric LUVs after exchange. The results show there was good agreement between outer leaflet charge determined by TMA-DPH and that estimated by TLC by assuming that donor phospholipid was transferred only into the outer leaflet. This was true both for vesicles with net negative, net positive, or near neutral outer leaflets. (As noted above, a near neutral outer leaflet was observed in the case of DOTAP:POPC out/POPG:POPC in/Chol vesicles, in which the amount of residual POPG in the outer leaflet was very high (Table 2).)

### Stability of Asymmetry

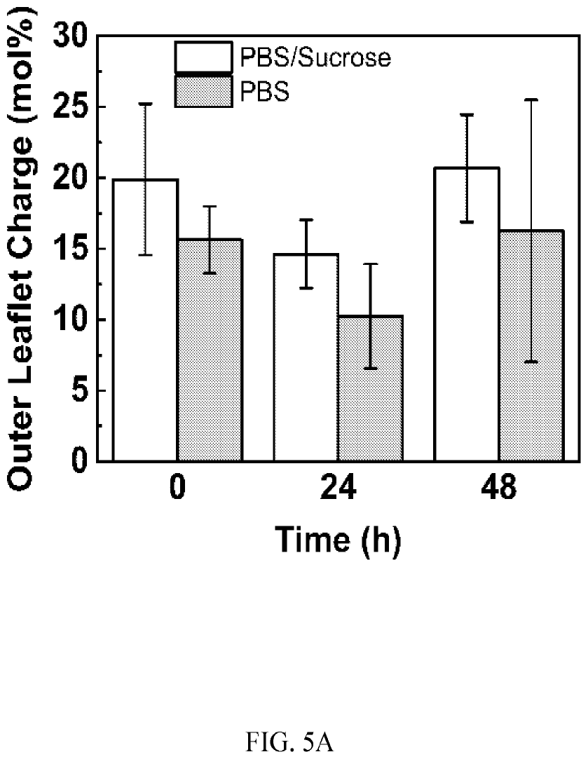

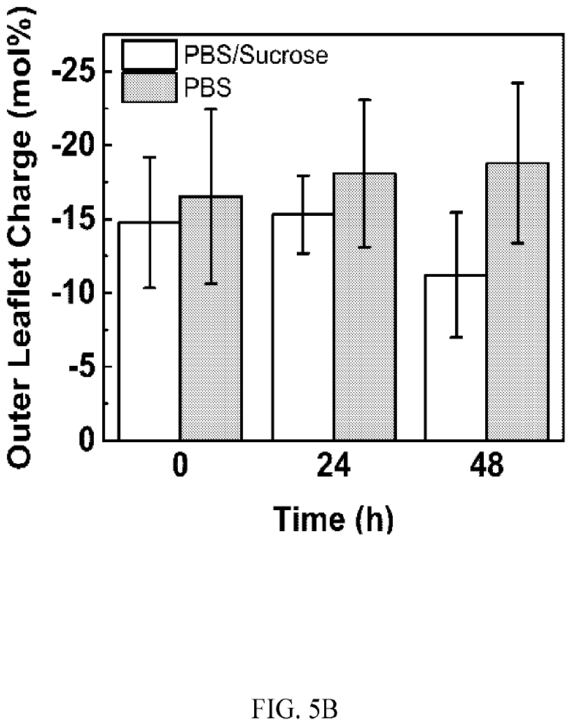

Although prior studies have demonstrated that lipid asymmetry is stable, often for days (See e.g., Cheng, H.-T.; London, E., Preparation and properties of asymmetric vesicles that mimic cell membranes effect upon lipid raft formation and transmembrane helix orientation. Journal of Biological Chemistry 2009, 284 (10), 6079-6092; Lin, Q.; London, E., Preparation of artificial plasma membrane mimicking vesicles with lipid asymmetry. PloS one 2014, 9 (1); Son, M.; London, E., The dependence of lipid asymmetry upon polar headgroup structure. Journal of lipid research 2013, 54 (12), 3385-3393; Dicorleto, P. E.; Zilversmit, D. B., Exchangeability and rate of flip-flop of phosphatidylcholine in large unilamellar vesicles, cholate dialysis vesicles, and cytochrome oxidase vesicles. Biochimica et Biophysica Acta (BBA)-Biomembranes 1979, 552 (1), 114-119; Johnson, L.; Hughes, M.; Zilversmit, D., Use of phospholipid exchange protein to measure inside-outside transposition in phosphatidylcholine liposomes. Biochimica et Biophysica Acta (BBA)-Biomembranes 1975, 375 (2), 176-185; and Rothman, J. E.; Dawidowicz, E. A., Asymmetric exchange of vesicle phospholipids catalyzed by the phosphatidylcholine exchange protein. Measurement of inside-outside transitions. Biochemistry 1975, 14 (13), 2809-2816), the TMA-DPH assay was used to confirm the stability of asymmetry. It is noteworthy that TLC would not show any change in overall phospholipid composition as asymmetry is lost, and so cannot be used to detect changes in the level of asymmetry. In contrast, the TMA-DPH assay directly measures outer leaflet charge, and so can be used to assay changes in asymmetry. Asymmetric LUVs were dispersed and incubated at room temperature in PBS/sucrose that was identical, and so osmotically balanced, with the solution inside the vesicles or dispersed and incubated in PBS. The outer leaflet charge of the asymmetric LUVs was measured by the TMA-DPH binding assay after 1 and 2 days. The results in FIG. 5 show behavior was similar when vesicles were dispersed in PBS or PBS with sucrose. The net mol % of charged lipids in the outer leaflet of both POePC:POPCout/POPS:POPC in/Chol and POPS:POPC out/POePC:POPC in/Chol asymmetric LUVs was relatively stable, not changing significantly in the first 48 h. This presumably reflects the low flip-flop rate of the lipids used.

### The Level and Stability of Doxorubicin Entrapment within Asymmetric LUVs

Next, the effect of lipid charge and asymmetry upon liposomal entrapment of the cationic anti-cancer drug doxorubicin (Dox) was measured. Dox intercalates between DNA base pairs, inhibiting topoisomerase II and thus replication (See e.g., Buchholz, T. A.; Stivers, D. N.; Stec, J.; Ayers, M.; Clark, E.; Bolt, A.; Sahin, A. A.; Symmans, W. F.; Hess, K. R.; Kuerer, H. M., Global gene expression changes during neoadjuvant chemotherapy for human breast cancer. The Cancer Journal 2002, 8 (6), 461-468; Hilmer, S. N.; Cogger, V. C.; Muller, M.; Le Couteur, D. G., The hepatic pharmacokinetics of doxorubicin and liposomal doxorubicin. Drug metabolism and disposition 2004, 32 (8), 794-799; and Tacar, O.; Sriamornsak, P.; Dass, C. R., Doxorubicin: an update on anticancer molecular action, toxicity and novel drug delivery systems. Journal of pharmacy and pharmacology 2013, 65 (2), 157-170). It is used encapsulated inside liposomes to lower its toxicity and prolong its circulation time (See e.g., Herman, E.; Rahman, A.; Ferrans, V.; Vick, J.; Schein, P., Prevention of chronic doxorubicin cardiotoxicity in beagles by liposomal encapsulation. Cancer Research 1983, 43 (11), 5427-5432; Gabizon, A. A., Selective tumor localization and improved therapeutic index of anthracyclines encapsulated in long-circulating liposomes. Cancer Research 1992, 52 (4), 891-896; Gabizon, A.; Catane, R.; Uziely, B.; Kaufman, B.; Safra, T.; Cohen, R.; Martin, F.; Huang, A.; Barenholz, Y., Prolonged circulation time and enhanced accumulation in malignant exudates of doxorubicin encapsulated in polyethylene-glycol coated liposomes. Cancer research 1994, 54 (4), 987-992; Bally, M. B.; Nayar, R.; Masin, D.; Hope, M. J.; Cullis, P. R.; Mayer, L. D., Liposomes with entrapped doxorubicin exhibit extended blood residence times. Biochimica et Biophysica Acta (BBA)-Biomembranes 1990, 1023 (1), 133-139; van Lummel, M.; van Blitterswijk, W. J.; Vink, S. R.; Veldman, R. J.; van der Valk, M. A.; Schipper, D.;

Dicheva, B. M.; Eggermont, A. M.; ten Hagen, T. L.; Verheij, M., Enriching lipid nanovesicles with short-chain glucosylceramide improves doxorubicin delivery and efficacy in solid tumors. The FASEB Journal 2011, 25 (1), 280-289; and Mayer, L. D.; Tai, L. C.; Ko, D. S.; Masin, D.; Ginsberg, R. S.; Cullis, P. R.; Bally, M. B., Influence of vesicle size, lipid composition, and drug-to-lipid ratio on the biological activity of liposomal doxorubicin in mice. Cancer research 1989, 49 (21), 5922-5930). Dox can cross lipid membranes, and be entrapped in their aqueous lumen by a pH-gradient (See e.g., Mayer, L.; Bally, M.; Cullis, P., Uptake of adriamycin into large unilamellar vesicles in response to a pH gradient. Biochimica Et Biophysica Acta (BBA)-Biomembranes 1986, 857 (1), 123-126; and Li, X.; Hirsh, D. J.; Cabral-Lilly, D.; Zirkel, A.; Gruner, S. M.; Janoff, A. S.; Perkins, W. R., Doxorubicin physical state in solution and inside liposomes loaded via a pH gradient. Biochimica et Biophysica Acta (BBA)-Biomembranes 1998, 1415 (1), 23-40) or by precipitation induced via a manganese-gradient (See e.g., Cheung, B. C.; Sun, T. H.; Leenhouts, J. M.; Cullis, P. R., Loading of doxorubicin into liposomes by forming Mn2+-drug complexes. Biochimica et Biophysica Acta (BBA)-Biomembranes 1998, 1414 (1-2), 205-216), a phosphate gradient (See e.g., Fritze, A.; Hens, F.; Kimpfler, A.; Schubert, R.; Peschka-Süss, R., Remote loading of doxorubicin into liposomes driven by a transmembrane phosphate gradient. Biochimica et biophysica acta (BBA)-biomembranes 2006, 1758 (10), 1633-1640), or a sulfate-gradient (See e.g., Bolotin, E. M.; Cohen, R.; Bar, L. K.; Emanuel, N.; Ninio, S.; Barenholz, Y.; Lasic, D. D., Ammonium sulfate gradients for efficient and stable remote loading of amphipathic weak bases into liposomes and ligandoliposomes. Journal of Liposome Research 1994, 4 (1), 455-479; and Haran, G.; Cohen, R.; Bar, L. K.; Barenholz, Y., Transmembrane ammonium sulfate gradients in liposomes produce efficient and stable entrapment of amphipathic weak bases. Biochimica et Biophysica Acta (BBA)-Biomembranes 1993, 1151 (2), 201-215). Liposome-entrapped ammonium sulfate has been used to aid stable entrapment of Dox. It was found that with 23% (w/w) sucrose entrapped inside the LUVs, the concentration of ammonium sulfate allowing entrapment of a high amount of Dox could be decreased to 50 mM (data not shown). Using these conditions, symmetric and asymmetric LUVs were prepared, and then the amount of Dox entrapped in the LUVs was assayed by measuring its fluorescence after washing the liposomes (See Methods).

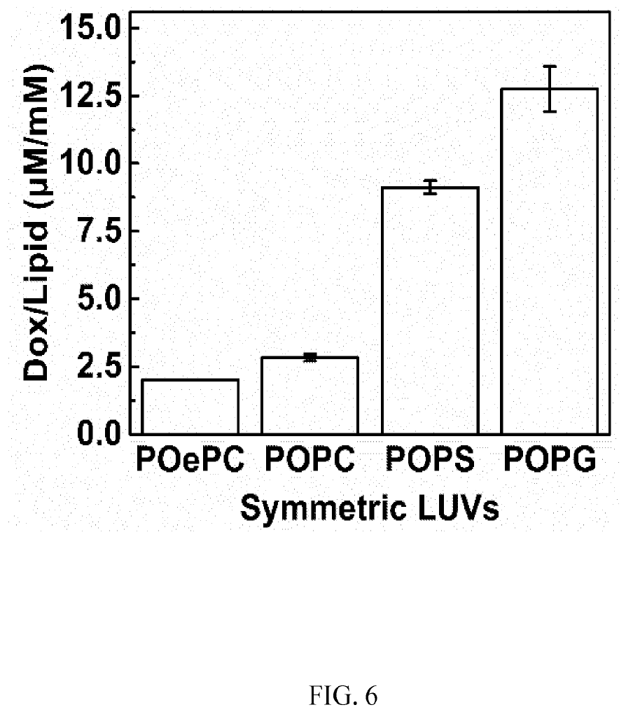

FIG. 6 shows the amount of Dox associated with symmetric LUVs in terms of the Dox/lipid ratio. LUVs were composed of either 60 mol % POPC and 40 mol % cholesterol, or of 15% POePC, POPS or POPG, 45 mol % POPC and 40 mol % cholesterol. The negatively charged LUVs (containing POPG and POPS) associated with 3-6 times more Dox than neutral (POPC) LUVs, or positively charged LUVs (containing POePC). This indicates that electrostatic interactions between lipids and Dox, has a strong influence on the amount of liposome-associated Dox.

Dox entrapment within symmetric anionic LUVs was highly stable. After pelleting samples and washing in PBS twice, 90 or more % of the initially trapped Dox remained in symmetric LUVs containing anionic phospholipid or lacking charged lipid. In contrast, symmetric LUVs containing cationic lipid only retained 60% of entrapped Dox under these conditions (Table 5).

Concentration of Dox and lipid in symmetric LUVs with entrapped Dox as a function of number of times pelleted. Sample name—1: initial preparation of LUVs with trapped Dox; Sample name—2 or 3: sample washed 1 or 2 times after initial preparation, respectively. Mean and standard deviation from three preparation is shown.

TABLE 5

| | Dox | Lipid | Dox/ipid |
|---|---|---|---|
| Symmetric LUVs | Concentration(μM) | Concentration(mM) | (μM/mM) |
| POePC:POPC/Chol -1 | 5.83 ± 0.16 | 1.72 ± 0.24 | 3.43 ± 0.41 |
| POePC:POPC/Chol -2 | 3.31 ± 0.33 | 1.38 ± 0.11 | 2.40 ± 0.14 |
| POePC:POPC/Chol -3 | 2.18 ± 0.32 | 1.08 ± 0.16 | 2.01 ± 0.01 |
| POPC/Chol -1 | 5.86 ± 0.14 | 1.92 ± 0.06 | 3.05 ± 0.06 |
| POPC/Chol -2 | 4.30 ± 0.16 | 1.53 ± 0.13 | 2.81 ± 0.14 |
| POPC/Chol -3 | 3.30 ± 0.11 | 1.17 ± 0.08 | 2.83 ± 0.12 |
| POPG:POPC/Chol -1 | 14.93 ± 0.52 | 1.18 ± 0.05 | 12.69 ± 0.33 |
| POPG:POPC/Chol -2 | 11.20 ± 0.35 | 0.88 ± 0.02 | 12.78 ± 0.66 |
| POPG:POPC/Chol -3 | 9.51 ± 0.22 | 0.75 ± 0.06 | 12.75 ± 0.83 |
| POPS:POPC/Chol -1 | 13.57 ± 0.47 | 1.35 ± 0.03 | 10.01 ± 0.12 |
| POPS:POPC/Chol -2 | 10.45 ± 0.40 | 1.19 ± 0.06 | 8.79 ± 0.13 |
| POPS:POPC/Chol -3 | 8.69 ± 0.47 | 0.95 ± 0.07 | 9.13 ± 0.24 |

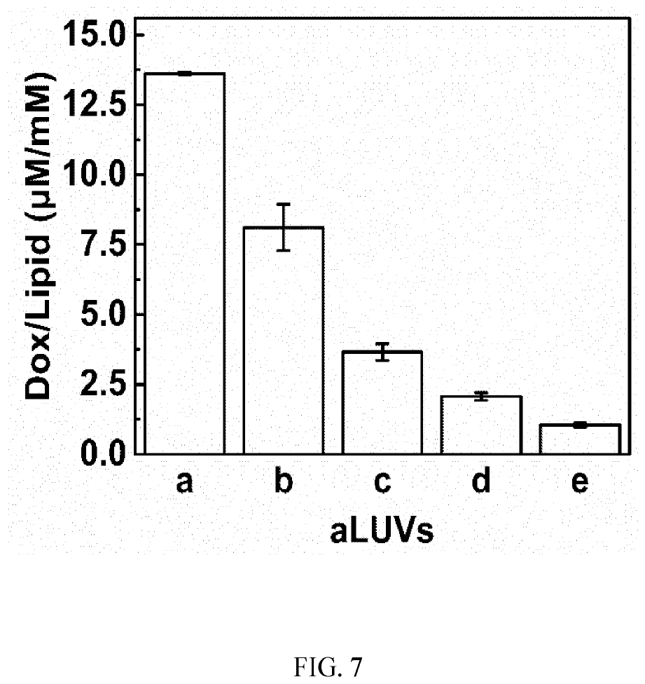

To determine how asymmetry of lipid charge would affect Dox association with liposomes these experiments were then repeated with asymmetric LUVs. As shown in FIG. 7, asymmetric vesicles with cationic POePC in their outer leaflets and anionic inner leaflets (compositions a and b) trapped the largest amount of Dox, in amounts per lipid similar to those in symmetric vesicles containing anionic lipids in both leaflets. In contrast, asymmetric LUVs with a similar overall lipid composition, but with the opposite asymmetry in which the inner leaflet was cationic and the outer leaflet was anionic (compositions d and e) trapped low amounts of Dox, similar to that trapped in symmetric vesicles containing cationic lipids in both leaflets. Compositions with a cationic outer leaflet and neutral inner leaflet (composition c) trapped an intermediate amount of Dox, similar to neutral symmetric vesicles.

These experiments demonstrate that the charge on the inner leaflet of a lipid vesicle determines how much Dox is trapped within the vesicle, with no appreciable effect of the outer leaflet lipid charge. The observation that vesicle outer leaflet charge has little effect implies it is very unlikely that significant amounts of Dox associate with the outer leaflet of the vesicles. The ability to control Dox entrapment by controlling the inner leaflet independently of the outer leaflet raises the possibility that asymmetric vesicles could have important advantages for drug delivery applications (see below).

### Discussion and Conclusion

Liposomal drug delivery is useful because liposomes can improve biodistribution, improve uptake by the target, and protect drugs from degradation, thus reducing side effects (See e.g., Sercombe, L.; Veerati, T.; Moheimani, F.; Wu, S. Y.; Sood, A. K.; Hua, S., Advances and challenges of liposome assisted drug delivery. Frontiers in pharmacology 2015, 6, 286). These advantages are affected by the intrinsic characteristics of the liposomes, such as the size of the liposomes, their net charge (or the zeta potential), and the selective binding properties of surface lipids (See e.g., Allen, T. M.; Cullis, P. R., Liposomal drug delivery systems: from concept to clinical applications. Advanced drug delivery reviews 2013, 65 (1), 36-48). In this this example, concentrated asymmetrically charged liposomes were prepared to further increase their utility. Asymmetric LUVs have been recently developed as natural membrane models to study the behavior and properties of membranes and membrane domains (See e.g. London, E., Membrane Structure—Function Insights from Asymmetric Lipid Vesicles. Accounts of Chemical Research 2019, 52 (8), 2382-2391; Marquardt, D.; Geier, B.; Pabst, G., Asymmetric lipid membranes: towards more realistic model systems. Membranes 2015, 5 (2), 180-196; and Kamiya, K.; Kawano, R.; Osaki, T.; Akiyoshi, K.; Takeuchi, S., Cell-sized asymmetric lipid vesicles facilitate the investigation of asymmetric membranes. Nature chemistry 2016, 8 (9), 881). However, highly asymmetrically charged LUVs have been little explored for drug delivery. In this report, cyclodextrin exchange was used to prepare asymmetric vesicles with various types of lipid charge asymmetry. It was found that LUVs with asymmetric charged leaflets could be prepared with a neutral inner leaflet, and positive or negative outer leaflet. It was also possible to prepare vesicles with a cationic inner leaflet and anionic outer leaflet and vice versa. Vesicles with one cationic and one anionic lipid leaflet were of particular interest because to our knowledge they have not been investigated in past studies. It was found that more than one type of anionic or cationic phospholipid could be used. Importantly, lipid asymmetry was stable, at least for 48 hours for the combinations of membrane lipids studied.

Asymmetric LUVs preparations may have several useful properties. One of the most important is increasing the concentration of drug that is trapped in the liposomes.

We found that for Dox, anionic lipid in the inner leaflet can maximize the amount and stability of drug entrapment within the vesicles. This may reflect an attraction of Dox to the anionic lipid surface during vesicle formation. This attraction might also prevent translocation of Dox across the membrane, and so inhibit leakage of Dox from the vesicles. In contrast, the charge on the outer leaflet had no influence upon the amount of Dox that was vesicle-associated. This indicates that it is very unlikely that there is very tight binding to a cationic surface or that significant amounts of Dox are associated with the outer leaflet of the vesicles.

It is possible that asymmetric LUVs with an anionic inner leaflet might be useful for drug Dox delivery. The dose of Dox that can be delivered without exhibiting cardiotoxicity is 10-50 fold less than with Doxil, which is liposome-encapsulated Dox (See e.g., Safra, T.; Muggia, F.; Jeffers, S.; Tsao-Wei, D.; Groshen, S.; Lyass, O.; Henderson, R.; Berry, G.; Gabizon, A., Pegylated liposomal doxorubicin (doxil):

reduced clinical cardiotoxicity in patients reaching or exceeding cumulative doses of 500 mg/m². Annals of Oncology 2000, 11 (8), 1029-1033; and Minotti, G.; Menna, P.; Salvatorelli, E.; Cairo, G.; Gianni, L., Anthracyclines: molecular advances and pharmacologic developments in antitumor activity and cardiotoxicity. Pharmacological reviews 2004, 56 (2), 185-229). Doxil does not contain anionic lipid. Thus, using asymmetric LUVs with an anionic inner leaflet, the dose of liposome-encapsulated Dox could potentially be increased several-fold without altering the outer leaflet lipid composition. High intra-liposomal drug-loading is an important parameter for its therapeutic application (See e.g., Drummond, D. C.; Noble, C. O.; Guo, Z.; Hong, K.; Park, J. W.; Kirpotin, D. B., Development of a highly active nanoliposomal irinotecan using a novel intraliposomal stabilization strategy. Cancer research 2006, 66 (6), 3271-3277; and Johnston, M. J.; Edwards, K.; Karlsson, G.; Cullis, P. R., Influence of drug-to-lipid ratio on drug release properties and liposome integrity in liposomal doxorubicin formulations. Journal of liposome research 2008, 18 (2), 145-157). It will be interesting to determine if similar principles can be used to optimize nucleic acid entrapment.

Asymmetric LUVs had additional properties that may be favorable for drug delivery. The presence of cholesterol significantly improved yield in many cases, and should be useful for reducing uptake of LUVs macrophages, which can clear liposomes from the circulation (See e.g., Allen, T.; Austin, G.; Chonn, A.; Lin, L.; Lee, K., Uptake of liposomes by cultured mouse bone marrow macrophages: influence of liposome composition and size. Biochimica et Biophysica Acta (BBA)-Biomembranes 1991, 1061 (1), 56-64). It should also be noted that the diameter of the asymmetric LUVs was ˜120 nm, which is a good size for drug delivery (See e.g., Nagayasu, A.;

Uchiyama, K.; Kiwada, H., The size of liposomes: a factor which affects their targeting efficiency to tumors and therapeutic activity of liposomal antitumor drugs. Advanced drug delivery reviews 1999, 40 (1-2), 75-87). For delivery to tumor tissues sizes in the range of 100-200 nm have been reported to be optimal for prolonging circulation time (See e.g., Litzinger, D. C.; Buiting, A. M.; van Rooijen, N.; Huang, L., Effect of liposome size on the circulation time and intraorgan distribution of amphipathic poly (ethylene glycol)-containing liposomes. Biochimica et Biophysica Acta (BBA)-Biomembranes 1994, 1190 (1), 99-107), increasing transfer from vascular to tumor tissue, (See e.g., Uchiyama, K.; Nagayasu, A.; Yamagiwa, Y.; Nishida, T.; Harashima, H.; Kiwada, H., Effects of the size and fluidity of liposomes on their accumulation in tumors: A presumption of their interaction with tumors. International journal of pharmaceutics 1995, 121 (2), 195-203; and Takakura, Y.; Takagi, A.; Hashida, M.; Sezaki, H., Disposition and tumor localization of mitomycin C-dextran conjugates in mice. Pharmaceutical research 1987, 4 (4), 293-300) accumulating around tumor tissue (See e.g., Papahadjopoulos, D.; Allen, T.; Gabizon, A.; Mayhew, E.; Matthay, K.; Huang, S.; Lee, K.; Woodle, M.; Lasic, D.; Redemann, C., Sterically stabilized liposomes: improvements in pharmacokinetics and antitumor therapeutic efficacy. Proceedings of the National Academy of Sciences 1991, 88 (24), 11460-11464; and Liu, D.; Mori, A.; Huang, L., Role of liposome size and RES blockade in controlling biodistribution and tumor uptake of GM1-containing liposomes. Biochimica et Biophysica Acta (BBA)-Biomembranes 1992, 1104 (1), 95-101), permeating through tumor capillaries (See e.g., Yuan, F.; Dellian, M.; Fukumura, D.; Leunig, M.; Berk, D. A.; Torchilin, V. P.; Jain, R. K., Vascular permeability in a human tumor xenograft: molecular size dependence and cutoff size. Cancer research 1995, 55 (17), 3752-3756; and Yuan, F.; Leunig, M.; Huang, S. K.; Berk, D. A.; Papahadjopoulos, D.; Jain, R. K., Mirovascular permeability and interstitial penetration of sterically stabilized (stealth) liposomes in a human tumor xenograft. Cancer research 1994, 54 (13), 3352-3356), retention in tumor interstitial spaces (See e.g., Huang, S.; Lee, K.; Hong, K.; Friend, D.; Papahadjopoulos, D., Microscopic localization of sterically stabilized liposomes in colon carcinoma-bearing mice. Cancer research 1992, 52 (19), 5135-5143; and Maeda, H.; Matsumura, Y., Tumoritropic and lymphotropic principles of macromolecular drugs. Critical reviews in therapeutic drug carrier systems 1989, 6 (3), 193-210), reducing the side effects relative to free drug (See e.g., Allen, T. M.; Cullis, P. R., Liposomal drug delivery systems: from concept to clinical applications. Advanced drug delivery reviews 2013, 65 (1), 36-48) reducing degradation by complement system (See e.g., Harashima, H.; Hiraiwa, T.; Ochi, Y.; Kiwada, H., Size Dependent Liposome Degradation in Blood: In vivo/In vitro Correlation by Kinetic Modeling. Journal of Drug Targeting 1995, 3 (4), 253-261; and Harashima, H.; Huong, T.; Ishida, T.; Manabe, Y.; Matsuo, H.; Kiwada, H., Synergistic effect between size and cholesterol content in the enhanced hepatic uptake clearance of liposomes through complement activation in rats. Pharmaceutical research 1996, 13 (11), 1704-1709) and uptakes by mononuclear phagocytes (See e.g., Nagayasu, A.; Uchiyama, K.; Kiwada, H., The size of liposomes: a factor which affects their targeting efficiency to tumors and therapeutic activity of liposomal antitumor drugs. Advanced drug delivery reviews 1999, 40 (1-2), 75-87). In the future, it will be important to test the entrapment of drugs or imaging reagents with both different charge and hydrophobicities, as well as the efficiency of drug delivery into cells as a function of outer leaflet composition and charge. Outer leaflet lipids can be optimized for slow clearance from the circulation and vesicle targeting (such as by using monosialoganglioside or polyethyleneglycol-binding-phospholipids) (See e.g., Gabizon, A.; Papahadjopoulos, D., Liposome formulations with prolonged circulation time in blood and enhanced uptake by tumors. Proceedings of the national academy of sciences 1988, 85 (18), 6949-6953; Woodle, M.; Matthay, K.; Newman, M.; Hidayat, J.; Collins, L.; Redemann, C.; Martin, F.; Papahadjopoulos, D., Versatility in lipid compositions showing prolonged circulation with sterically stabilized liposomes. Biochimica et Biophysica Acta (BBA)-Biomembranes 1992, 1105 (2), 193-200) and to release drug most effectively at certain sites (such as by using pH-sensitive lipid C12-200) (See e.g., Love, K. T.; Mahon, K. P.; Levins, C. G.; Whitehead, K. A.; Querbes, W.; Dorkin, J. R.; Qin, J.; Cantley, W.; Qin, L. L.; Racie, T., Lipid-like materials for low-dose, in vivo gene silencing. Proceedings of the National Academy of Sciences 2010, 107 (5), 1864-1869). Manipulating outer leaflet charge by adjusting the donor lipid composition should itself be important, since the net charge of the outer leaflet of LUVs should alter unfavorable binding to charged surfaces and biomolecules, which can play a role in immunogenicity, screening by spleen or kidney, accumulation in the liver, and cytotoxicity (See e.g., Fröhlich, E., The role of surface charge in cellular uptake and cytotoxicity of medical nanoparticles. International journal of nanomedicine 2012, 7, 5577). Outer leaflet charge should also influence drug concentration at the target, which when optimized would reduce drug side effects.

Aspects of Example I are further described in Li, B.; London, E., Preparation and Drug Entrapment Properties of Asymmetric Liposomes Containing Cationic and Anionic Lipids, Langmuir 2020, 36, 42, 12521-12531 (herein entirely incorporated by reference).

### Example 2

Numbers of therapeutic methods have been developed to regulate the protein expression to treat diseases in human body. Small molecule drugs, as with natural compounds, are old functional models for therapeutic protein modification, inhibition, activation, upregulation and degradation. Even though some of the small molecule drugs are very effective and selective, most of them have very high off-target rates on either proteins or cells with undesirable side effects and lots of the proteins are difficult to target. As an alternative approach, direct protein delivery with therapeutic purpose would be more effective and practical, such as the insulin, already injected into patients to treat diabetes. However, the effective cases of protein delivery are limited due to the difficulties of correct localizing of nucleus, cytoplasmic, and transmembrane proteins to replace dysfunctional endogenous proteins, and the short lives, requires multiply times of dosing or large dosage.

Gene therapy has been an effective alternative method of endogenously regulating the protein production in cells, as experiments have confirmed that certain proteins can be expressed, appropriately post-transcription modified and correctly intracellular localized via delivering nucleic acids (DNA or RNA) into the cell nucleus. However, the progress of prompting the work from in vitro to in vivo is quite slow, mainly because of the tough barriers of gene delivery, such as the serum nuclease, tissue distribution, cellular uptake, endosomal escape and delivery into the nucleus, so that very few gene therapy treatments have been successful in clinical stages.

With the success of Pfizer messenger RNA (mRNA) vaccine, mRNA delivery and the whole gene delivery area have drawn numerous attentions recently. DNA or mRNA delivery is able to express specific protein, with necessary post-translation modification and cellular localization. Scientists can selectively express any endogenous or exogenous protein virtually via DNA or RNA delivery, which allows doctors to treat countless diseases and disorders with potential much fewer side effects than the small molecule drugs used right now. Even though the DNA or RNA based therapeutic method has great potential, very few of them have been developed. The main reason is not because of the efficiency of the DNA or RNA itself since the in vitro success has been proved multiply times, but because of the obstacles of systemically delivering DNA or RNA into the disease cell in vivo.

The first barrier for DNA or RNA delivery is its stability in plasma, because DNA or RNA can be degraded by exonuclease and endonuclease circulating in the plasma. Even though some chemical modification can be helpful for protecting DNA or RNA from nuclease, they are not practical since the DNA or RNA are synthesized enzymatically via replication or transcription. These chemical modifications can also lower the efficiency of transcription or translation. Alternative strategies of protecting DNA or RNA while circulating in the plasma are in need. Beside degradation, the next barrier of DNA or RNA delivery faces is biodistribution. Most of the intravenously injected unmodified DNA or RNA are found accumulated in the liver, while they need to be presented in the specific organ tissue to be function. If this issue can be resolved via manipulating mRNA delivery targeting to different specific issues, DNA or RNA delivery would be applied for clinical treatment of a myriad of diseases.

With appropriate protection and location, the next barrier of DNA or RNA delivery would be cellular uptake. The heavily negative charge of the phosphorus backbone of nucleic acids would not allow it travel through the hydrophobic region of the plasma membrane by itself. It has been discovered that cell can take up materials from its surrounding environment via some ways, such as macropinocytosis, caveolae-mediated endocytosis, clathrin mediated endocytosis, etc. But most cells would not take up DNA or RNA freely. An appropriate delivery vehicle, which can merge onto the cell membrane or bind to the surface receptor, would significantly improve the cellular uptake of DNA or RNA. After being endocytosed, the delivery vehicle must help DNA or RNA to escape the endosome to enter the cytoplasm to enter the nucleus to transcript, to bind to mRNA to inhibition translation, or to bind to the ribosomal complex for expression. The negative charged nature of mRNA makes it difficult to cross the endosome membrane, so some disruptions induced by the delivery vehicle are helpful. If DNA or RNA fails to escape the endosome or enters the lysosome, it will be digested by nucleases in late endosomes and lysosomes.

To achieve gene therapy via in vivo DNA or RNA delivery, lots of vehicles and methods have been developed and used, such as nanoparticles, ligand conjugates and liposomal nanoparticles. None of them are prefect in every aspect, but some have been shown to be better and more practical than others. Viral particles are a potential method to deliver mRNA into specific cell types in vivo. However, the clearance of the viral particles by circulating nuclease and pattern recognition, cytokine cascade induced by the innate defensive mechanism and immunostimulation, and the difficulty of large-scale manufacture process largely limit its further clinical application. Ligand conjugates are proved to be useful as delivering DNA or RNA into specific cell types in vivo via covalent attachment to cellular receptor. But they cannot protect DNA or RNA from circulating nuclease and assist DNA or RNA escaping the late endosomes, which largely undermines their practicality. Thus, a vector is needed to entrap, protect, and shuttle the DNA or RNA payload across the cell membrane to enable their access to the cytosol to elicit their function.

Liposomal mRNA delivery has not been fully developed and there is great potential for improvement. Liposomes can form unilamellar vesicles or multilamellar complexes with DNA or RNA molecules, which protect the DNA or RNA in a safe stable aqueous environment from all nucleases and plasma proteins, and maintain DNA or RNA reactivity without any chemical modification. Additionally, the vesicle can be modified to circumvent host immune system to avoid lots of side effects, including immunogenicity and toxicity. Also, delivery efficiency can be maintained for repeat dosing.

When delivered by liposomes, the in vivo biodistribution of DNA or RNA will be primarily determined by the size, charge and molecular composition of liposomes. Liposomes, are typically 50-200 nm in diameter, which prevents clearance from kidney whose vascular fenestration size is 20-30 nm. They can easily penetrate and transfect epithelial cells of the liver, spleen and must tumor tissues. Additionally, the liposomes with modified ligands on their surface are capable of binding to the cell surface to facilitate cellular uptake via active targeting. Also, binding to some plasma proteins, such as apolipoproteins could facilitate liposomal cellular endocytosis via binding to the cell surface receptors. No matter what pathway is used for cellular uptake, most liposomes enter the cell via entry into endosomes. There are different pathways for the liposomal entrapped mRNA to escape the endosomes and enter the cytoplasm. One is using ionizable lipid as a proton sponge. After protonation, more counter ions would enter the endosome causing it to swell and rupture. Another pathway is that the ionizable lipids turn into cationic charge in acidic late endosome and thus bind and merge into the endosomal membrane to destabilize the membrane so that the entrapped DNA or RNA could escape and enter the cytoplasm.

From the perspective of future manufacture and clinical applications, the liposomal DNA or RNA delivery has more advantages. Liposomes can be self-assembling with DNA or RNA entrapped automatically by electrostatic interaction with cationic or ionizable lipid molecules. The size, surface charge, reaction property, stability and the biodistribution of liposomes are all mainly dependent on the lipid composition instead of mRNA sequence entrapped inside, which makes manufacture process scalable and reproducible, and enables the development of liposomal delivery to specific cells or tissues much easier via using same lipid composition and different DNA or RNA. Last but not least, liposomes have been proven to be very stable, maintaining their size and entrapment ability at 37° C. even after months storage, prolonging their shelf life and enabling easy transportation and storage.

The efficacy of liposomal delivery can be varied by modulating lipid composition through many ways. Cholesterol concentration has a significant effect on liposomal stability and promoting the lamellar and H_{II} phase, which in turn affects the drug entrapment and release from liposomes. The mole percent of lipid-anchored PEG can modulate the size and circulation time of liposomes, and reduce hemolysis and clearance by the immune system. Even a small change of 0.5 mol % of lipid-anchored PEG can result in a very big different delivery efficacy by an order of magnitude. The lipid composition and the ratio of ionizable lipid to DNA or RNA have been optimized for mRNA delivery to maximize the therapeutic window. The liposomal DNA or RNA delivery is very promising as an alternative method of gene therapy to express therapeutic proteins inside human body, and it has the great potential of improving for highly desired therapeutic effects with lowered dose via overcoming every barrier listed above.

However, PEG lipid can take up lots of space inside, which might seriously affect the DNA or RNA entrapment efficiency of the liposomes and ionizable lipids inside needs acidic environment to be protonated to be positively charged to entrap negatively charged nucleic acids. After the nucleic acid entrapment, the liposomes will be in the physiological environment and the ionizable lipids, not only outside ones but also inside ones, will turn into neutral or a little negative charge. Then the entrapment of DNA or RNA based on the electrostatic interaction might be affected and become unstable to lower the therapeutic efficiency. To solve this problem to achieve maximized therapeutic efficiency with maximized entrapment and delivery efficiency and lower drug dose needed, asymmetric liposomes may be provided with permanent positive charged lipids inside, and ionizable lipids and PEG lipids outside via preparing positively charged liposomes with DNA or RNA entrapped inside first and exchanging ionizable and PEG lipids onto the outside lipids. Here, we will use siRNA-sized DNA as entrapment nucleic acid model.

### Materials and Methods

### Materials

1-palmitoyl-2-oleoyl-sn-glycero-3-phosphocholine (POPC), 1-palmitoyl-2-oleoyl-sn-glycero-3-ethylphosphocholine (chloride salt) (POePC), 1,2-dioleoyl-3-trimethylammonium-propane (chloride salt) (DOTAP), 1-palmitoyl-2-oleoyl-sn-glycero-3-phospho-(1′-rac-glycerol) (sodium salt) (POPG), 1-palmitoyl-2-oleoyl-sn-glycero-3-phospho-L-serine (sodium salt) (POPS), and cholesterol (Choi) were purchased from Avanti Polar Lipids (Alabaster, AL). Lipids were stored in chloroform at −20° C. Concentrations were determined by dry weight. High performance thin layer chromatography (HP-TLC) plates (Silica Gel 60) were purchased from VWR International (Batavia, IL). Methyl-α-cyclodextrin (MαCD) was purchased from AraChem Cyclodextrin Shop (Tilburg, the Netherlands). It was dissolved in distilled water at close to 300 mM, and then filtered through a Sarstedt (Numbrecht, Germany) 0.2 μm pore syringe filter. The exact concentration of MαCD was determined by comparing the refractive index of the solutions to a standard curve of refractive index vs. MαCD concentration for a known amount of MαCD dissolved in a known final volume of solution. 1(4-trimethylammoniumphenyl)-6-phenyl-1,3,5-hexatriene p-toluenesulfonate (TMADPH) was purchased from the Molecular Probes (Eugene, OR) division of Invitrogen (Carlsbad, CA). DNA (Alex647-5′-CGGGTGGGAGCAGATCTTATTGAG-3′ (SEQ ID NO: 1) and 5′-CGGGTGGGAGCAGATCTTATTGAG-3′) (SEQ ID NO: 2) was purchased from Integrated DNA Technologies (Coralville, Iowa). 1,6-diphenyl-1,3,5-hexatriene (DPH) was purchased from Sigma-Aldrich (St. Louis, MO). PBS (10× phosphate-buffered saline, diluted to 1×: 10 mM sodium phosphate; and 150 mM sodium chloride, pH ˜7.4) was purchased from Bio-Rad (Hercules, CA).

### Preparation of Symmetric LUV

Prior to vesicle preparation, the initial lipid concentrations were measured by gravimetric analysis of the stock solutions. Lipids dissolved in chloroform were mixed in glass tubes, dried under a warm nitrogen stream and subjected to high vacuum for 1 h. The dried lipid mixtures were dispersed to 8 mM lipid concentration with 23% (w/w) sucrose in 0.83×PBS (sucrose/PBS, ˜1009 mOsm, prepared by dissolving sucrose in 1×PBS). For DNA entrapment, lipid mixtures were dispersed in sucrose/PBS with 500 μM DNA (10% of the DNA has been labeled with Alexa647). The samples were vortexed briefly and then incubated at 37° C. for 15 min. The lipid mixtures were then cooled to room temperature and subjected to seven cycles of freeze-thaw in a liquid nitrogen bath, alternating with a 27° C. water bath. To form LUVs of uniform vesicle size, the lipid mixtures were then extruded 11 times through 100 nm-pore polycarbonate membranes (Sigma-Aldrich, St. Louis, MO).

If needed to wash away external sucrose (e.g. to prepare acceptor vesicles for lipid exchange or prepare samples for size measurements), 200 μL aliquots of LUV were mixed with 3.8 mL 1×PBS (˜325 mOsm) and pelleted by ultracentrifugation at 190,000×g for 30 min at 23° C. using a Beckman L8-80M ultracentrifuge with a SW-60 rotor. Following pelleting, the supernatant was removed, the LUV containing-pellet dispersed in 0.5 mL PBS. When samples had entrapped DNA they were re-centrifuged twice with 4 mL PBS using the same protocol. Finally, the LUV pellet was dispersed in 500 μL PBS, covered with aluminum foil, and reserved for use. Unless otherwise noted samples were used within 2 h of preparation.

### Preparation of Donor Lipid-Loaded MαCD for Lipid Exchange Experiments

Desired ratios of charged lipids (POePC, DOTAP, POPS, or POPG) and zwitterionic POPC dissolved in chloroform were combined in glass tubes, dried under a warm nitrogen stream, and then subjected to high vacuum for 1 h. The dried lipids were placed in a 70° C. water bath and dispersed at 70° C. with an aliquot of pre-warmed PBS, and then an aliquot of pre-warmed MαCD, to give a final concentration of 40 mM MαCD and 16mM lipid. The samples were vortexed briefly, and then vortexed in a multitube vortexer for 2 h at 55° C., cooled to room temperature, covered in foil, and reserved for further use.

### Preparation of Acceptor LUV for Lipid Exchange Experiments

Desired ratios of charged lipids (POePC, DOTAP, POPS, or POPG), zwitterionic POPC, and cholesterol (40 mol % of total lipid) dissolved in chloroform were combined in glass tubes. LUVs were then prepared as described above for symmetric vesicles.

### Outer Leaflet Lipid Exchange

To wash away untrapped sucrose from acceptor LUVs, 500 μL aliquots of acceptor LUVs were diluted with 3.5 ml PBS and subjected to ultracentrifugation at 190,000 g for 30 min at 23° C. as above. The supernatant was discarded, the LUV pellets were resuspended to 8 mM lipid concentration with PBS and used immediately. To exchange the outer leaflet of acceptor LUVs, 500 μL of the donor lipid-MαCD mixture and 500 μL of the acceptor LUVs mixtures were combined, covered in foil, and shaken for 45 min at 37° C. These lipid-exchange mixtures were layered over 3 mL 7.4% (w/w) sucrose dissolved in 3.76×PBS (prepared by dissolving sucrose in 4×PBS, ˜1448 mOsm) and subjected to ultracentrifugation at 190,000×g for 45 min at 23° C. Following centrifugation, most of the supernatant was carefully removed, leaving approximately 750 μL sucrose/4×PBS and loosely pelleted asymmetric LUVs in the bottom of the centrifuge tube. The upper portion of the tube was swabbed with a clean, dry cotton tipped applicator to remove residual adhering donor lipids and MαCD. Approximately 3.25 mL PBS was then added to the tube and thoroughly mixed with asymmetric LUVs and residual supernatant. This mixture was centrifuged a second time as above for 30 min. Following centrifugation, all remaining supernatant was removed, and the pellet was dispersed for immediate use in up to 500 μL PBS or distilled water if samples were for fluorescence analysis. The asymmetric LUVs lipid concentration was determined by DPH assay (see below in Methods) and the mean yield was ˜10.5% of theoretical maximal yield), with a final lipid concentration 0.83±0.22 mM. Concentration of entrapped DNA is determined by the fluorescence of Alexa647.

### Fluorescence Measurements

Fluorescence measurements were carried out using a SPEX FluoroLog 3 spectrofluorometer (Horiba Scientific, Edison, New Jersey) using quartz semimicro cuvettes (excitation pathlength, 10 mm; emission pathlength, 4 mm) TMADPH fluorescence was measured at an excitation wavelength of 364 nm and emission wavelength of 426 nm. For the DNA concentration measurement, dissolving the vesicles with 1% Triton first and then Alexa647 fluorescence was measured at an excitation wavelength of 648 nm and emission wavelength of 677 nm. The slit bandwidths were set to 3 mm (about 5 nm bandpass) for both excitation and emission.

Fluorescence was measured at room temperature. Background samples, which lacked fluorescent probe, had negligible intensity (<1% of samples with fluorescent probe).

Lipid concentration in asymmetric LUVs and symmetric LUVs after centrifugation was estimated via the level of DPH bound as measured by fluorescence.

DPH fluorescence was measured at an excitation wavelength of 358 nm and emission wavelength of 430 nm. A standard linear curve of fluorescence vs. lipid concentration was prepared using symmetric LUVs with POPC, 40 mol % cholesterol and with or without 15 mol % of the charged lipids used in the LUVs to be assayed. (However, it should be noted that the standard curves were not affected by the presence or absence of 15 mol % charged phospholipid, and so were averaged to give the final standard curve.) Standard samples were diluted with PBS to the desired concentration.

### Results and Discussion

### The Level of DNA Entrapment within Asymmetric LUVs

The effect of lipid charge and asymmetry upon liposomal entrapment of the anionic DNA (for gene therapy) was measured. Since negative charged nucleic DNA can used for gene delivery to express some specific genes, it has many benefits to study liposomal DNA entrapment. Also, the DNA entrapment studies could be a very good model for RNA entrapment studies since they have very similar size, structure, and charge, and RNA is more fragile and expensive for in vitro liposomal entrapment studies. In our studies we prepared symmetric and asymmetric LUVs, and then the amount of DNA entrapped in the LUVs was assayed by measuring the Alexa647 fluorescence attached on it after washing the liposomes (See Methods).

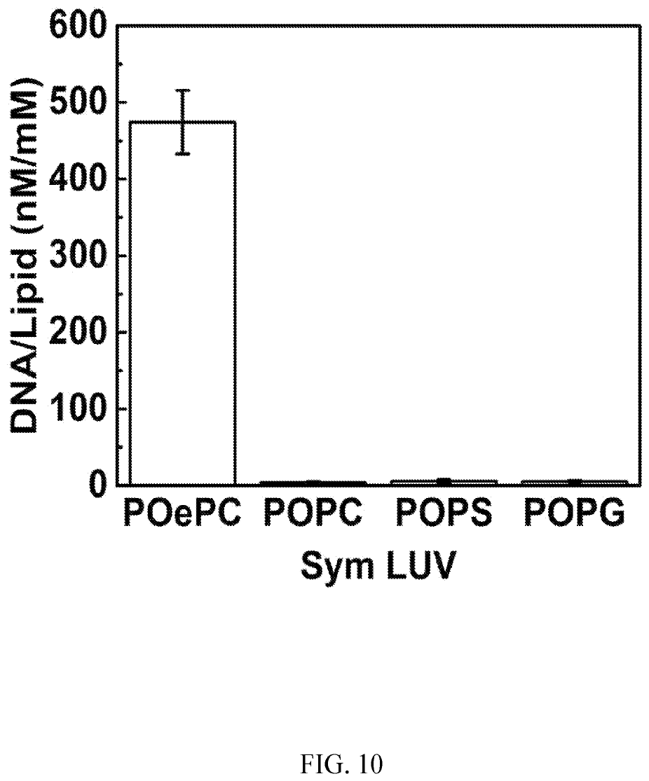

FIG. 10 shows the amount of DNA associated with symmetric LUVs in terms of the DNA/lipid ratio. LUVs were composed of either 60 mol % POPC and 40 mol % cholesterol, or of 15% POePC, POPS or POPG, 45 mol % POPC and 40 mol % cholesterol. The positively charged LUVs (containing POePC) associated with 100 times more DNA than neutral (POPC) LUVs, or negatively charged LUVs (containing POPS or POPG). This indicates that electrostatic interactions between lipids and DNA, has a strong influence on the amount of liposome-associated DNA.

More specifically, FIG. 10 depicts DNA entrapment within symmetric LUVs containing 40 mol % cholesterol and either 60 mol % POPC or 45 mol % POPC and 15 mol % POePC, POPS or POPG. These samples were pelleted by centrifugation and washed twice to match the protocol used for asymmetric vesicles (see Methods). Results show mean values and standard deviations from three vesicle preparations.

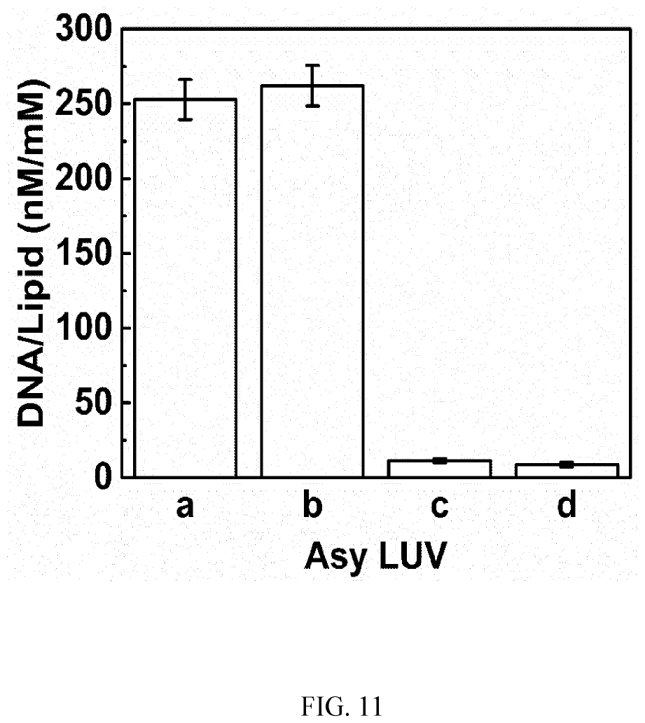

To determine how asymmetry of lipid charge would affect DNA association with liposomes, these experiments were then repeated with asymmetric LUVs. As shown in FIG. 11, asymmetric vesicles with cationic POePC in their inner leaflets and cationic POPS or POPG in their inner leaflets (compositions a and b) trapped the largest amount of DNA, in amounts per lipid similar level to those in symmetric vesicles containing anionic lipids in both leaflets even though they have 2 times difference. In contrast, asymmetric LUVs with the neutral lipid in the inner leaflet, and cationic POePC or anionic POPS in the outer leaflet, (compositions c and d) trapped low amounts of DNA, similar to that trapped in symmetric vesicles containing anionic or neutral lipids in both leaflets.

More specifically, FIG. 11 depicts DNA entrapment within asymmetric LUVs. (a) POPS:POPC out/POePC:POPC in/Chol, (b) POPG:POPC out/POePC:POPC in/Chol, (c) POePC:POPC out/POPC in/Chol, (d) POPS:POPC out/POPC in/Chol. Results show mean values and standard deviations from three vesicle preparations.

These experiments demonstrate that the charge on the inner leaflet of a lipid vesicle determines how much DNA is trapped within the vesicle, with no appreciable effect of the outer leaflet lipid charge. The observation that vesicle outer leaflet charge has little effect implies it is very unlikely that significant amounts of DNA associate with the outer leaflet of the vesicles. The ability to control DNA entrapment by controlling the inner leaflet independently of the outer leaflet raises the possibility that asymmetric vesicles could have important advantages for drug delivery applications.

The entire disclosure of all applications, patents, and publications cited herein are herein incorporated by reference in their entirety. While the foregoing is to directed to embodiments of the present disclosure, other and further embodiments of the disclosure may be devised without departing from the basic scope thereof.

1. A charged vesicle, comprising:
a bilayer of lipids forming a shell, wherein the bilayer of lipids comprises an inner layer of lipids and an outer layer of lipids, wherein the inner layer of lipids and the outer layer of lipids are different, and wherein the bilayer is characterized by having an asymmetric charge distribution; and
an interior portion of the shell configured to entrap a drug or biomolecule.

2. The charged vesicle of claim 1, wherein the inner layer of lipids has a first net charge and the outer layer of lipids have a second net charge different than the first net charge.

3. The charged vesicle of claim 2, wherein the first net charge is positive, and the second net charge is negative.

4. The charged vesicle of claim 2, wherein the first net charge is negative, and the second net charge is positive.

5. The charged vesicle of claim 1, wherein the drug or biomolecule has a positive or negative charge.

6. The charged vesicle of claim 1, wherein the inner layer is negative, and the drug is positive, and a leakage of the drug is reduced compared to a non-charged vesicle comprising a same drug.

7. The charged vesicle of claim 1, wherein the inner layer is positive and the drug is negative, and a leakage of the drug is reduced compared to a non-charged vesicle comprising a same drug.

8. The charged vesicle of claim 1, wherein the interior portion comprises an aqueous medium.

9. The charged vesicle of claim 1, wherein the biomolecule is a negatively charged DNA or RNA.

10. The charged vesicle of claim 1, wherein the drug is doxorubicin.

11. The charged vesicle of claim 1, wherein the inner layer of lipids has a neutral charge and the outer layer of lipids has a second net charge which is positive or negative.

12. The charged vesicle of claim 1, wherein the inner layer of lipids and outer layer of lipids each comprise charged phospholipids in an amount of 25-50% of each layer of lipids.

13. The charged vesicle of claim 1, wherein the inner layer and the outer layer further comprise cholesterol.

14. The charged vesicle of claim 1, wherein the inner layer of lipids and outer layer of lipids each comprise a mixture of one or more uncharged lipids, one or more cationic lipids, or one or more anionic lipids.

15. The charged vesicle of claim 1, wherein the inner layer of lipids and the outer layer of lipids comprise a mixture of two uncharged lipids, one or more of two cationic lipids, and one or more of three anionic lipids.

16. The charged vesicle of claim 15, wherein the two uncharged lipids are cholesterol and zwitterionic lipid.

17. The charged vesicle of claim 16, wherein the zwitterionic lipid is phosphatidylcholine (Popc).

18. The charged vesicle of claim 15, wherein the two cationic lipids comprise O-ethyl phosphatidyl choline or dioleoyl-3-trimethylammonium propane.

19. The charged vesicle of claim 15, wherein the three anionic lipids comprise phosphatidylglycerol, phosphatidylserine, and phosphatidic acid.

20. A method for preparing a large unilamellar vesicle (LUV), comprising: contacting a cyclodextrin-lipid complex comprising one or more charged donor lipids and methyl-α-cyclodextrin with a liposome comprising a unilamellar membrane having an inner leaflet and an outer leaflet, to exchange one or more charged donor lipids from the cyclodextrin-lipid complex to the outer leaflet to form an asymmetrical large unilamellar vesicle.

21. The method of claim 20, further comprising forming a cyclodextrin-lipid complex with one or more preselected ratios of charged and uncharged lipids.

22. The method of claim 21, wherein the charged lipids comprise one or more of 1 palmitoyl-2-oleoyl-sn-glycero-3-ethylphosphocholine (POePC), 1,2-dioleoyl-3-triethylammonium-propane (chloride salt) (DoTAP), 1-palmitoyl-2-oleoyl-sn-glycero-3-phospho-L-serine (sodium salt) (POPS), 1-palmitoyl-2-oleoyl-sn-glycero-3-phospho-(1′-rac-glycerol) (sodium salt) (POPG), or 1-palmitoyl-2-oleoyl-sn-glycero-3-phosphate-L-serine (sodium salt) (POPA).

23. The method of claim 22, wherein the charged lipids are selected from a group consisting of 1 palmitoyl-2-oleoyl-sn-glycero-3-ethylphosphocholine (POePC), 1,2-dioleoyl-3-triethylammonium-propane (chloride salt) (DoTAP), 1-palmitoyl-2-oleoyl-sn-glycero-3-phospho-L-serine (sodium salt) (POPS), 1-palmitoyl-2-oleoyl-sn-glycero-3-phospho-(1′-rac-glycerol) (sodium salt) (POPG), or 1-palmitoyl-2-oleoyl-sn-glycero-3-phosphate-L-serine (sodium salt) (POPA), and combinations thereof.

24. The method of claim 20, wherein the liposome comprising a unilamellar membrane having an inner leaflet and an outer leaflet comprises a preselected ratio of charged lipids and cholesterol.

25. The method of claim 24, wherein the charged lipids comprise one or more of 1 palmitoyl-2-oleoyl-sn-glycero-3-ethylphosphocholine (POePC), 1,2-dioleoyl-3-triethylammonium-propane (chloride salt) (DoTAP), 1-palmitoyl-2-oleoyl-sn-glycero-3-phospho-L-serine (sodium salt) (POPS), 1-palmitoyl-2-oleoyl-sn-glycero-3-phospho-(1′-rac-glycerol) (sodium salt) (POPG), or 1-palmitoyl-2-oleoyl-sn-glycero-3-phosphate-L-serine (sodium salt) (POPA).

26. The method of claim 20, wherein contacting further comprises incubating the cyclodextrin-lipid complex and the liposome in a solution under conditions such that a plurality of lipids are exchanged between the cyclodextrin-lipid complex and the outer leaflet in an amount sufficient to provide a net charge to the outer leaflet that is opposite of the net charge of the inner leaflet.

27. The method of claim 26, wherein incubating occurs for a duration between 30 minutes and 2 hours.

28. The method of claim 20, wherein the lipid is an unnatural lipid or comprises a label.

29. The method of claim 28, wherein the label is selected from a group consisting of a fluorescent dye and a radioisotope.

30. The method of claim 20, further comprising forming a multilamellar vesicle comprising at least one lipid prior to forming said cyclodextrin-lipid complex.

31. The method of claim 30, wherein forming said cyclodextrin-lipid complex comprises incubating said multilamellar vesicle with a solution comprising a cyclodextrin.

32. The method of claim 31, wherein said incubation occurs at about 37° C. for about 30 minutes.

33. A kit for substituting lipids in a unilamellar vesicle to form an asymmetric unilamellar vesicle, comprising: at least one α-cyclodextrin; at least one first instruction for forming a cyclodextrin-lipid complex including the at least one lipid bound to the α-cyclodextrin; and at least one second instruction describing a method for using the at least one cyclodextrin-lipid complex to exchange the at least one lipid between a lipid bilayer of a liposome membrane and the cyclodextrin-lipid complex to form an asymmetric unilamellar vesicle.
